SEQUENCE LISTING (44 sequences)

SEQ ID NO: 1 - DNA, 22 bp, Artificial Sequence
  Chemically Synthesized
agattctaccgaagagacggac

SEQ ID NO: 2 - DNA, 21 bp, Artificial Sequence
  Chemically Synthesized
cgtcgggaccacattgatgta

SEQ ID NO: 3 - DNA, 23 bp, Artificial Sequence
  Chemically Synthesized
atgagtgctattcgtgatggaga

SEQ ID NO: 4 - DNA, 19 bp, Artificial Sequence
  Chemically Synthesized
tgggccaatttgcctgctt

SEQ ID NO: 5 - DNA, 21 bp, Artificial Sequence
  Chemically Synthesized
ccagggtgattgacaggtttg

SEQ ID NO: 6 - DNA, 23 bp, Artificial Sequence
  Chemically Synthesized
ccagttttctaatgctacccact

SEQ ID NO: 7 - DNA, 23 bp, Artificial Sequence
  Chemically Synthesized
gattctgagcaaaatagccagca

SEQ ID NO: 8 - DNA, 21 bp, Artificial Sequence
  Chemically Synthesized
ggcttccttcttgttgtgtgt

SEQ ID NO: 9 - DNA, 21 bp, Artificial Sequence
  Chemically Synthesized
ccactagggctgatactggct

SEQ ID NO: 10 - DNA, 19 bp, Artificial Sequence
  Chemically Synthesized
gaggcgggtggttgactac

SEQ ID NO: 11 - DNA, 22 bp, Artificial Sequence
  Chemically Synthesized
gttgtccaccttcgttactcag

SEQ ID NO: 12 - DNA, 23 bp, Artificial Sequence
  Chemically Synthesized
gagaatggcagatcatcccaaat

SEQ ID NO: 13 - DNA, 21 bp, Artificial Sequence
  Chemically Synthesized
agtggagtttgaccccttacc

SEQ ID NO: 14 - DNA, 21 bp, Artificial Sequence
  Chemically Synthesized
gaagtgcctctcggagtcgta

SEQ ID NO: 15 - DNA, 19 bp, Artificial Sequence
  Chemically Synthesized
cgagcgttgggcgacttta

SEQ ID NO: 16 - DNA, 21 bp, Artificial Sequence
  Chemically Synthesized
caggagcatgaactcgtcctc

SEQ ID NO: 17 - DNA, 23 bp, Artificial Sequence
  Chemically Synthesized
tacttggaaaatcacagttcccg

SEQ ID NO: 18 - DNA, 20 bp, Artificial Sequence
  Chemically Synthesized
cggcacaggttgtcactctc

SEQ ID NO: 19 - DNA, 21 bp, Artificial Sequence
  Chemically Synthesized
gtcctgcgtctgcaagaagag

SEQ ID NO: 20 - DNA, 20 bp, Artificial Sequence
  Chemically Synthesized
ctcgcattcgttgctgtagg

SEQ ID NO: 21 - DNA, 21 bp, Artificial Sequence
  Chemically Synthesized
tgctctcacctcatcagcagt

SEQ ID NO: 22 - DNA, 22 bp, Artificial Sequence
  Chemically Synthesized
cacaactcctccaatcacaact

SEQ ID NO: 23 - DNA, 22 bp, Artificial Sequence
  Chemically Synthesized
ttggacattctcgctatctcct

SEQ ID NO: 24 - DNA, 21 bp, Artificial Sequence
  Chemically Synthesized
agtgctttgatctgttccgtc

SEQ ID NO: 25 - DNA, 22 bp, Artificial Sequence
  Chemically Synthesized
ctcagaagctgggttggtttat

SEQ ID NO: 26 - DNA, 22 bp, Artificial Sequence
  Chemically Synthesized
tttatcgaggatgtcacgttgg

SEQ ID NO: 27 - DNA, 23 bp, Artificial Sequence
  Chemically Synthesized
agaaaaggtgacctagacaaagc

SEQ ID NO: 28 - DNA, 21 bp, Artificial Sequence
  Chemically Synthesized
ccttgtagcagcacccaatct

SEQ ID NO: 29 - DNA, 21 bp, Artificial Sequence
  Chemically Synthesized
ggtgggcactaatacaactgg

SEQ ID NO: 30 - DNA, 22 bp, Artificial Sequence
  Chemically Synthesized
cacacagaataaacggcaggta

SEQ ID NO: 31 - DNA, 23 bp, Artificial Sequence
  Chemically Synthesized
ctgatgcaggaactgtatagcac

SEQ ID NO: 32 - DNA, 20 bp, Artificial Sequence
  Chemically Synthesized
cacagcgtctagcacctctt

SEQ ID NO: 33 - DNA, 20 bp, Artificial Sequence
  Chemically Synthesized
cctgcaaggaatcaggggac

SEQ ID NO: 34 - DNA, 22 bp, Artificial Sequence
  Chemically Synthesized
gtcaagcattcgtagctcttct

SEQ ID NO: 35 - DNA, 19 bp, Artificial Sequence
  Chemically Synthesized
agcgggatcgtgaccagat

SEQ ID NO: 36 - DNA, 22 bp, Artificial Sequence
  Chemically Synthesized
tgaccttgcctctccacttatc

SEQ ID NO: 37 - DNA, 23 bp, Artificial Sequence
  Chemically Synthesized
ttggcatcttctattgcctcatc

SEQ ID NO: 38 - DNA, 21 bp, Artificial Sequence
  Chemically Synthesized
tgtcgcaacttgtattggtcc

SEQ ID NO: 39 - DNA, 21 bp, Artificial Sequence
  Chemically Synthesized
aacgtgcccttgtttgtccaa

SEQ ID NO: 40 - DNA, 21 bp, Artificial Sequence
  Chemically Synthesized
gagtccttcacccggatcgta

SEQ ID NO: 41 - DNA, 19 bp, Artificial Sequence
  Chemically Synthesized
gcgctgggtatgcgatctc

SEQ ID NO: 42 - DNA, 19 bp, Artificial Sequence
  Chemically Synthesized
cagcctgccttaggggaag

SEQ ID NO: 43 - DNA, 23 bp, Artificial Sequence
  Chemically Synthesized
agccgtcagggatgtactataac

SEQ ID NO: 44 - DNA, 23 bp, Artificial Sequence
  Chemically Synthesized
agggaatcatttggctctgtaga

(19) United States
(12) Patent Application Publication
(10) Pub. No.: US 2017/0327874 A1
(43) Pub. Date: Nov. 16, 2017

(54) Methods of Diagnosing Bacterial Infections

(71) Applicant: University of Rochester, Rochester, NY (US)

(72) Inventors: Ann R. Falsey, Rochester, NY (US); Thomas J. Mariani, Pittsford, NY (US); Edward E. Walsh, Pittsford, NY (US); Derick Peterson, Pittsford, NY (US)

(21) Appl. No.: 15/592,445

(22) Filed: May 11, 2017

Related U.S. Application Data
(60) Provisional application No. 62/334,828, filed on May 11, 2016.

(51) Int. Cl. C12Q 1/68

(52) U.S. Cl. CPC C12Q 1/689; C12Q 2600/16; C12Q 2600/118; C12Q 2600/158

(57) ABSTRACT

The present invention relates to compositions and methods useful for the assessment, diagnosis, and treatment of bacterial infections.

### CROSS-REFERENCE TO RELATED APPLICATIONS

This application claims priority to and the benefit of U.S. Provisional Patent Application No. 62/334,828, filed May 11, 2016, the content of which is incorporated in its entirety herein.

### STATEMENT REGARDING FEDERALLY SPONSORED RESEARCH OR DEVELOPMENT

This invention was made with government support under HHSN272201200005C awarded by National Institutes of Health. The government has certain rights in the invention.

### BACKGROUND OF THE INVENTION

Lower respiratory tract infections (LRTI) occur commonly throughout life, accounting for substantial morbidity and mortality in adults. In most cases the precise microbial etiology of LRTI is unknown and antibiotics are administered empirically. Although sensitive molecular diagnostics such as polymerase chain reaction (PCR) allow clinicians to rapidly and accurately diagnose a wide variety of respiratory viruses, their impact on patient management and antibiotic prescription has been modest primarily due to concern about bacterial co-infection. Such concerns are not unfounded as it was recently found that approximately 30% of hospitalized adults with viral LRTI had evidence of concomitant bacterial infection. The study of bacterial lung infection has been hampered by insensitive tests for invasive disease and difficulty interpreting sputum culture results. Blood cultures are positive in only 10-15% of pneumonia cases and sputum is often contaminated with upper airway flora. Clinical parameters such as fever, purulent sputum, white blood cell count and radiographic patterns do not provide sufficient precision to reliably distinguish viral from bacterial infections. Thus, “ruling out” bacterial respiratory infection is extremely difficult resulting in a default position of prescribing antibiotics to most patients hospitalized with LRTI. This practice results in significant antibiotic overuse with resultant adverse effects and increased healthcare costs. Recently, serum biomarkers such as procalcitonin have shown some promise as a supplement to clinical judgment in assessing patients with LRTI but need for more accurate tests remains.

Gene expression profiling of peripheral blood mononuclear cells (PBMCs) as well as whole blood represents a powerful new approach for analysis of the host response during infection. Preliminary studies indicate that viruses and bacteria trigger specific host transcriptional patterns in blood yielding unique “biosignatures” that may discriminate viral from bacterial causes of infection. Ramilo and colleagues used gene array analysis on extracted RNA from small volumes of blood from young children with febrile illnesses to differentiate infection with bacteria from viruses or virus plus bacteria, and also between gram-positive and gram-negative bacterial infection (Ramilo, 2007, Blood 109:2066-77). Another study conducted gene expression microarray analyses blood samples from 30 febrile children positive for viral or bacterial infection and 22 afebrile controls (. Blood leukocyte transcriptional profiles distinguished virus-positive children from both virus-negative afebrile controls and children with bacterial infection (Hu et al., 2013, PNAS 110:12792-7). Another study recently identified ten classifier genes in adults hospitalized with LRTI that discriminated between bacterial and viral infection. Eight of the ten classifier genes were interferon (IFN) related genes, which were over expressed in viral infection and absent in bacterial infection (Suarex et al., 2015, J Infect Dis 212:213-22).

There is thus a need in the art for compositions and methods for diagnosing a bacterial infection in a subject. The present invention addresses this unmet need in the art.

### SUMMARY OF THE INVENTION

In some aspects, the present invention provides a method for treatment of a respiratory infection in a subject. In one embodiment, the method comprises step of administering to the subject, who has been identified as having a differentially expressed level of one or more biomarkers selected from one or more biomarkers set forth in Table 1, an effective amount of an antibiotic.

In one embodiment, the one or more biomarkers is an RNA biomarker. In one embodiment, one or more biomarkers is selected from the group consisting of ICAM1, ITGAL, ITGB2, PECAM1, FADS2, PLA2GA4, CTSG, IGFBP2, IGFBP6, MMP2, ACOX3, and any combination thereof.

In one embodiment, the respiratory infection is a lower respiratory tract infection. In one embodiment, the antibiotic is one or more selected from Amoxicillin, Ampicillin, Cloxacillin, Dicloxacillin, Nafcillin, Oxacillin, Penicillin G, Penicillin V, Piperacillin, Cefadroxil (cefadroxyl), Cefalexin (cephalexin), Cefalotin (cephalothin), Cefapirin (cephapirin), Cefazolin (cephazolin), Cefradine (cephradine), Cefaclor, Cefotetan, Cefoxitin, Cefprozil (cefproxil), Cefuroxime, Cefdinir, Cefixime, Cefotaxime, Cefpodoxime, Ceftizoxime, Ceftriaxone, Ceftazidime, Cefepime, Ceftobiprole, Ceftaroline, Aztreonam, Imipenem, Imipenem, cilastatin, Doripenem, Meropenem, Ertapenem, Azithromycin, Erythromycin, Clarithromycin, Dirithromycin, Roxithromycin, Clindamycin, Lincomycin, Amikacin, Gentamicin, Tobramycin, Ciprofloxacin, Levofloxacin, Moxifloxacin, Trimethoprim-Sulfamethoxazole, Doxycycline, Tetracycline, Vancomycin, Teicoplanin, Telavancin, and Linezolid.

In another aspect, the present invention provides a method of diagnosing a bacterial infection in a subject. In one embodiment, the method comprises detecting the level of one or more biomarkers in a biological sample obtained from the subject, wherein the one or more biomarkers is selected from one or more biomarkers set forth in Table 1; comparing the level of the one or more biomarkers in the biological sample to a control level of the one or more biomarkers; and diagnosing the subject with a bacterial infection when the one or more biomarkers is differentially expressed in the biological sample as compared to the control level.

In one embodiment, the one or more biomarkers is selected from the group consisting of ICAM1, ITGAL, ITGB2, PECAM1, FADS2, PLA2GA4, CTSG, IGFBP2, IGFBP6, MMP2, ACOX3, and any combination thereof.

In one embodiment, the biological sample is selected from the group consisting of blood, plasma, saliva, and urine. In one embodiment, the subject is diagnosed when the one or more biomarkers is increased as compared to the control level.

In another aspect, the invention provides a kit for diagnosing a bacterial infection. In one embodiment the kit comprises a reagent for measuring the level of one or more biomarkers in a biological sample of a subject, wherein the one or more biomarkers is selected from one or more biomarkers set forth in Table 1.

In one embodiment, the one or more biomarkers is selected from the group consisting of ICAM1, ITGAL, ITGB2, PECAM1, FADS2, PLA2GA4, CTSG, IGFBP2, IGFBP6, MMP2, ACOX3, and any combination thereof. In one embodiment, the biological sample is selected from the group consisting of blood, urine, saliva and plasma.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1D, depicts results from experiments depicting transcriptomics data quality metrics. Shown here are total number of input reads (FIG. 1A), percentage of input reads mapped to the genome (FIG. 1B), and the proportion of the genome for which transcripts were detected (FIG. 1C), for each individual sample (FIG. 1D) Shown here are the RNA concentrations of blood collected in Tempus tubes (circles) and CPT tubes (squares).

FIG. 2 depicts violin plots of RNASeq-based expression data for each of the 10 genes. Q-values for differential expression between bacterial and non-bacterial groups are indicated beneath the gene names. Horizontal lines indicate group medians.

FIG. 3 depicts gene set enrichment analysis plot for these 10 genes. Seven genes (vertical black lines) are contained in the leading edge of the enrichment plot.

FIG. 4 depicts results from experiments validating previously identified markers: Quantitative reverse transcriptase-polymerase chain reaction (qPCR)- based expression data for each of the 10 genes. P-values for differential expression between bacterial and non-bacterial groups are indicated beneath the gene names. Horizontal lines indicate group medians.

FIG. 5, comprising FIG. 5A and FIG. 5B, depicts 141 gene global expression patterns in LRTI. FIG. 5A: Shown is a heat map for the 141 genes (rows) demonstrating significantly different expression levels between bacterial and non-bacterial groups. Each column represents an individual subject, and subjects were grouped based upon microbiological diagnosis, using independent hierarchical clustering (Euclidean distance with average linkage). FIG. 5B: The same data are presented, grouped by clinical diagnosis. The pink semi-circle denotes the patient with influenza A and a single blood culture positive for S. aureus.

FIG. 6 depicts Ingenuity Pathway Analysis (IPA) which was used to identify enriched functions based on the nonbacterial (numerator) vs. bacterial (denominator) comparison at a q <0.01 comprising of 1434 genes (left) and q <0.01, up-regulated 85 genes (right). The rows indicate functions where the Benjamini-Hochberg corrected p-values were <0.01 for the input gene list. Bar length corresponds to the —log10 corrected p-value. Orange/blue indicate increased/decreased function, respectively, based on the activation z-score which measures the degree to which coordinate changes of the function's constituent genes are consistent with its activity.

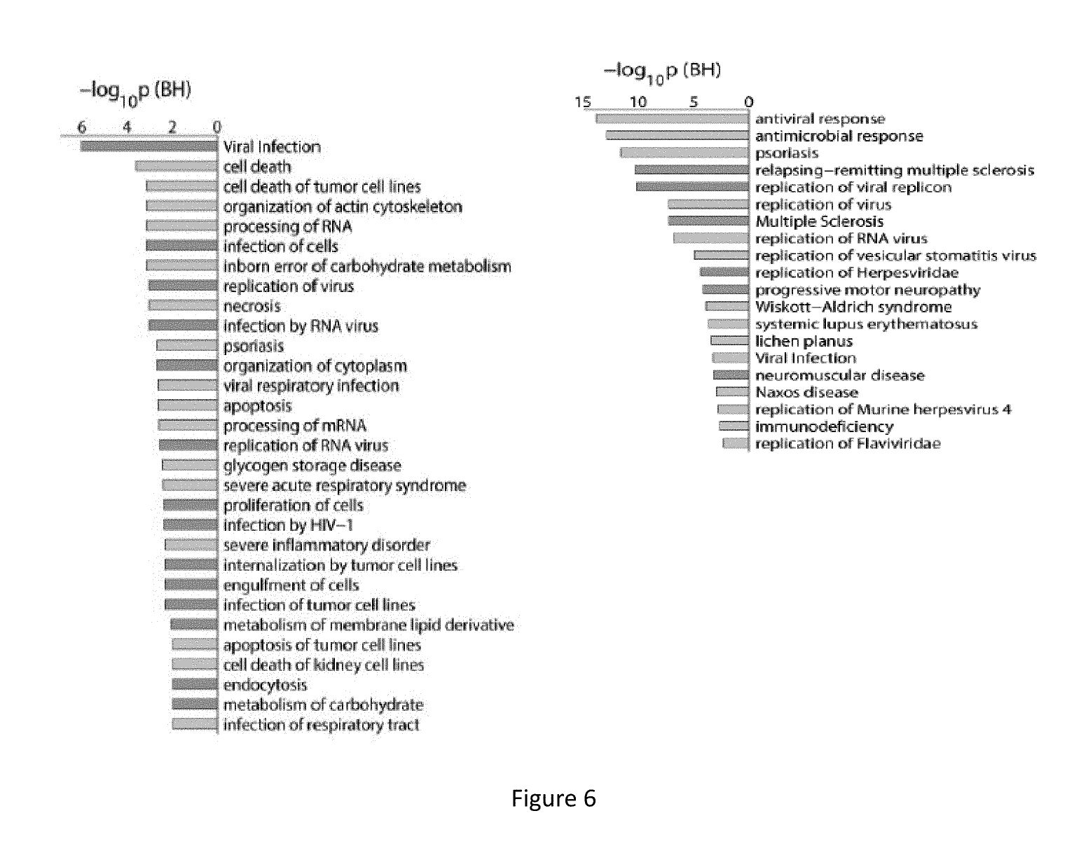

FIG. 7 depicts experiments where IPA was used to identify enriched canonical pathways using the same gene lists. Nine pathways are shown where the Benjamini-Hochberg corrected p-values were less than 0.05 for 1434 genes at q<0.01 (top) and 3 pathways are shown where the Benjamini-Hochberg corrected p-values were less than 0.05 for 85 up-regulated genes at q less than 0.01 (bottom).

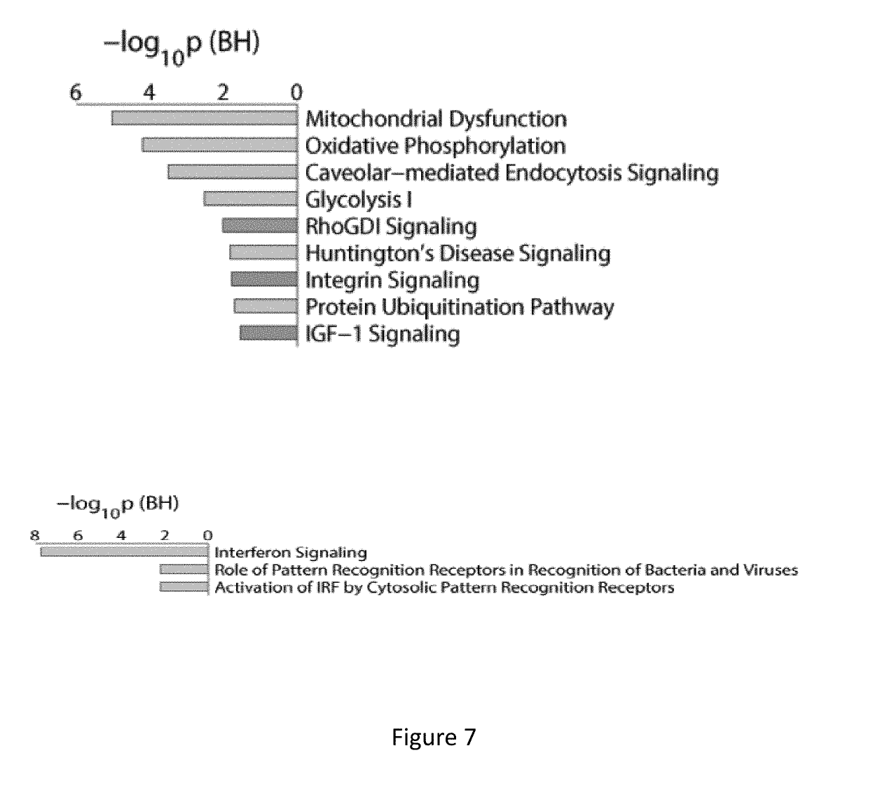

FIG. 8 depicts the upstream regulators where the —log10 of the overlap p-value was >2.5. Bars are colorized if there was a predicted activation state based on the pattern of direction of change of the targets.

FIG. 9 depicts a subset of genes (out of the 141 genes, significant by Wilcoxon Rank Test at Bonferroni p<0.05) selected for their magnitude difference or biological relevance.

FIG. 10 depicts the validation of univariate markers: Quantitative reverse transcriptase polymerase chain reaction (qPCR) was used to confirm expression patterns of genes identified by Wilcoxon Rank test (Bonferroni FDR<0.05). Nine genes were selected, of which eight showed significant difference between bacterial and viral groups by Mann-Whitney U test. Shown are individual sample fold changes (on a logarithmic scale) for each gene, in both groups. Shown are p-values from MWU test.

FIG. 11 depicts expression of multivariate markers in RNAseq data. Eleven genes were identified as expression markers capable of distinguishing bacterial from non-bacterial LRTI using a pathway-based supervised PCA and screened LASSO analysis. Shown are violin plots of data for 11 genes by sample group. Horizontal line segments are group medians. q-values for the non-bacterial (viral) vs. bacterial (viral+bacterial and bacterial) are indicated beneath the gene names

FIG. 12 depicts validation of multivariate markers: Quantitative reverse transcriptase polymerase chain reaction (qPCR) was used to confirm expression patterns of genes identified as predictors. Eleven genes were selected, of which six showed significant difference between bacterial and viral groups by Mann-Whitney U test. Shown are individual sample fold changes (on a logarithmic scale) for each gene, in both groups. Gene names appended with # indicate non-significant difference by MWU.

FIG. 13 depicts results from example experiments, showing Area Under the ROC Curve (AUC) characteristics for fully nested cross-validated estimates using the “pathway”-selected 11 gene set, the “array”-selected 10 gene set, and the 4 clinical variables. These data indicate that the pathway-based 11-gene predictor outperforms both the clinical and array-based gene models.

FIG. 14 depicts enriched canonical pathways represented by genes identified as significantly differentially expressed at varying levels of significance (BH-corrected q). Sixteen significant pathways are shown where the corrected p-values were <0.05 for at least one gene listed.

FIG. 15 depicts a heat map for the 10 predictive genes identified by pathway analysis as predictive of bacterial infection (rows) demonstrating differential expression in the 3 groups, bacterial, mixed viral bacterial and viral alone. Each column represents an individual subject.

FIG. 16, comprising FIG. 16A and FIG. 16B, depicts a Comparison of Predictive Performance for Gene Expression and Clinical Biomarkers. FIG. 16A. LASSO-penalized logistic regression retained 3 pathways consisting of a total of 11 genes providing the greatest predictive value for classifying subjects as bacterial or non-bacterial. *LASSO Pathway OR are odds ratios per SD of the hard-thresholded 1st PC of the nominally significant genes within the pathway. ***Constrained Gene OR=exp (Gene Loading * log (LASSO Pathway OR)/SDPathway)=(LASSO Pathway OR)(Gene Loading/(SD of Pathway)). FIG. 16B. Cross-validation chose a 4-predictor model consisting of nasal congestion, infiltrates on chest radiograph, blood urea nitrogen levels and white blood cell count.

FIG. 17 depicts a flow chart of the analysis of different gene sets derived from RNA sequencing data from 94 subjects with confirmed bacterial-involved or non-bacterial LRTI selected for RNAseq interrogation. First, the expression of 10 genes identified by Suarez et. al. was assessed to be differentially expressed comparing bacterial to viral infections. Next, the 141 most differentially-expressed genes were identified, as defined by statistical differences in bacterial vs. non-bacterial LRTI. Separately, a pathway-based approach was used to develop a novel gene expression classifier for discriminating bacterial vs. non-bacterial LRTI. * An additional 9 genes of biologic interest were selected from the 141gene set after considering the 10 Suarez genes. Validation of RNAseq-based expression estimates for selected genes, of particular biological interest, was attempted by qPCR. Finally, performance of the novel 3 pathway-based 11 gene classifier was assessed in the cohort.

FIG. 18 depicts the functional assessment of expression of biomarkers. Ingenuity Pathway Analysis (IPA) was used to identify enriched biological functions. represented by the 141 genes demonstrating significantly different expression levels between bacterial and non-bacterial groups, or subgroups of these genes, as described. The gene sets are: q<0.01: n=1434; q<0.005: n=1051; q<0.001: n=433; q<0.0005: n=304; q<0.01, up-regulated only: n=85. Gene sets are listed in columns, functions/pathways are listed in rows, and significance is displayed as the —log10 Benjamini-Hochberg (B-H) corrected p-value. Orange/blue circles indicate increased/decreased function, respectively, based on the activation z-score which measures the degree to which coordinate changes of the function's constituent genes are consistent with its activity.

FIG. 19 depicts the global expression patterns in LRTI restricted to patients with Staphylococcal bacteremia. Shown is a heat map for the 141 genes (rows) demonstrating significantly different expression levels between bacterial and non-bacterial groups. Each column represents an individual subject with the orange Xs at top of columns indicating patients with bacterial classification and the pink * indicating viral-bacterial classification (influenza A and methicillin resistant staphylococcal aureus) The black semi-circle at the top denotes the patient misclassified by the predictor genes as nonbacterial.

FIG. 20 depicts the clinical characteristics of misclassified subjects. NOD129 Patient history of asthma/COPD, presented with 3-4 days of wheezing, dyspnea and increased thick yellow phlegm. Good quality sputum grew 3+H. influenzae. Treated with antibiotics and recovered. NABO6D Patient with history of asthma/COPD, recent sick contacts, presented with 2 days of fever, dyspnea and productive cough. CXR was without infiltrate. Patient tested positive for influenza A and good quality sputum grew 4+ M. catarrhalis. Patient was treated with oseltamivir and antibiotics and recovered. NAB3B7 patient was a 73 year-old woman with a history of oxygen dependent COPD. She had been hospitalized 6 days' prior with productive cough, wheezing and difficulty breathing. She had a negative influenza PCR at that time and adequate sputum that showed gram negative diplococci on Gram stain and grew 4+ Haemophilus influenzae and was discharged on moxifloxacin and a steroid taper for presumed AECOPD. She returned to the hospital complaining of increased difficulty breathing with wheezing and a dry cough. On exam, she had a normal blood pressure with temperature of 37.4 and was noted to have diffuse wheezing and respiratory distress. Oxygen saturation was 88% on 2 liters of oxygen and chest radiograph showed a possible infiltrate in the left lower lobe. Peripheral white blood cell count was 16,800 with 2% bands and 2% atypical lymphocytes. Nasal swab was PCR positive for influenza A H1N1 and one set of blood cultures on admission grew MRSA. Oseltamivir, Cefepime and flagyl were administered for possible healthcare associated pneumonia. Two additional sets of blood cultures drawn prior to receipt of vancomycin were negative. She was treated with 2 weeks of IV vancomycin and prednisone and gradually improved. NC08A0 Patient presented with severe sore throat, fever, body aches, dizziness, cough and confusion. Rapid strep test was positive for Group A streptococcus (GAS). CXR showed a patchy infiltrate in the right lung base. Sputum after antibiotics grew 1+ GAS. Patient received antibiotics and recovered. N94C54 Patient with end stage renal disease, COPD and CHF presented with 2 days of dry cough, sore throat and dyspnea. Felt to have primarily CHF exacerbation. No antibiotics given. N7BFA3 Patient with asthma presented with 1 day of nasal congestion, fever, dyspnea and wheezing with green sputum production. Husband sick with similar illness. Patient had patchy infiltrate in the right lung base and good quality sputum that grew only normal respiratory flora and blood cultures were negative. She was treated with antibiotics for community acquired pneumonia and recovered. N7F9AA Patient present presented with 1 day of scratchy throat, dry cough, dyspnea with fever and myalgia. CXR was without infiltrate. Patient was tested positive for influenza A and was treated with oseltamivir without antibiotics and recovered. NB3035 Patient with coronary artery disease, CHF and tobacco and alcohol abuse presented with nasal congestion for several days followed by increasing dyspnea with cough productive of foamy blood streaked sputum. CXR showed edema and sputum culture was negative for pathogens. Impression from the cardiologist was viral bronchitis induced CHF. The hospitalist was concerned for pneumonia and the patient received antibiotics and recovered. N8323B Patient with asthma presented with 2 days of nasal congestion, hoarseness, fever, wheezing and yellow sputum. CXR was negative for infiltrate and sputum grew normal flora. She received antibiotics and recovered. NF73CC Patient with CHF presented with several days of nasal congestion, sore throat, fevers and then developed a cough productive of yellow sputum. CXR showed no infiltrate, sputum grew normal flora. Antibiotics were given initially then stopped due to low suspicion of bacterial infection and he recovered. NA240D Patient with history of heroin abuse and COPD presented with headache, body aches, chills and dyspnea. CXR showed basilar atelectasis. Patient tested positive for influenza and was treated with oseltamivir. Antibiotics were given initially then stopped due to low suspicion of bacterial infection and she recovered. NA8BE9 Patient with asthma presented with 4 days of chills, wheezing and cough. CXR was negative and sputum grew normal flora but patient received antibiotics and recovered. N45520 Patient with asthma presented with 3 days of wheezing, dyspnea and clear sputum. CXR was clear, no antibiotics were given and patient recovered

### DETAILED DESCRIPTION

The present invention relates to the discovery that the expression patterns of certain genes or otherwise expression levels of some biomarkers are associated with bacterial infections. Thus, in various embodiments described herein, the methods of the invention relate to methods of diagnosing a subject as having a bacterial infection, methods for differentiating a viral infection from a bacterial infection, methods of treating a condition and methods of altering treatment in a subject.

In some embodiments, biomarkers associated with a bacterial infection are up-regulated, while in other embodiments, the biomarkers associated with a bacterial infection are down-regulated. Thus, the invention relates to compositions and methods useful for the detection and quantification of biomarkers, including RNA, protein and microRNA biomarkers, for the diagnosis, assessment, and characterization of bacterial infection in a subject in need thereof, based upon the expression level of at least one biomarker or an expression pattern of at least one biomarker that is associated with bacterial infection.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Antisense,” as used herein, refers to a nucleic acid sequence which is complementary to a target sequence, such as, by way of example, complementary to a target mRNA, miRNA or snoRNA sequence, including, but not limited to, a mature target miRNA sequence, or a sub-sequence thereof. Typically, an antisense sequence is fully complementary to the target sequence across the full length of the antisense nucleic acid sequence.

“Complementary” as used herein refers to the broad concept of subunit sequence complementarity between two nucleic acids. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

As used herein, an “immunoassay” refers to any binding assay that uses an antibody capable of binding specifically to a target molecule to detect and quantify the target molecule.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

As used herein, the term “diagnosis” refers to the determination of the presence of a disease or disorder, such as a bacterial infection. In some embodiments of the present invention, methods for making a diagnosis are provided which permit determination of the presence of a disease or disorder, such as bacterial infection.

The terms “dysregulated” and “dysregulation” as used herein describes a decreased (down-regulated) or increased (up-regulated) level of expression of a mRNA, miRNA, snoRNA, or protein present and detected in a sample obtained from subject as compared to the level of expression of that mRNA, miRNA snoRNA, or protein present in a comparator sample, such as a comparator sample obtained from one or more normal, not-at-risk subjects, or from the same subject at a different time point. In some instances, the level of mRNA, miRNA snoRNA, or protein expression is compared with an average value obtained from more than one not-at-risk individuals. In other instances, the level of mRNA, miRNA snoRNA, or protein expression is compared with a mRNA, miRNA level snoRNA, or protein level assessed in a sample obtained from one normal, not-at-risk subject.

As used herein, the terms “therapy” or “therapeutic regimen” refer to those activities taken to alleviate or alter a disorder or disease, such as a respiratory infection, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

An “effective amount” as used herein, means an amount which provides a therapeutic or prophylactic benefit.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease, such as a respiratory or bacterial infection, being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder, such as a respiratory infection, experienced by a subject.

As used herein, “Differentially expressed level” refers to a group of biomarkers having differentially decreased expression level, or a differentially elevated expression level. It also means that in a group of biomarkers some biomarkers have differentially decreased expression and some biomarkers have differentially elevated expression.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a comparator.

“Differentially decreased expression” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments therebetween than a comparator.

As used herein “endogenous” refers to any material from or produced inside an organism, cell, tissue or system.

The term “expression” as used herein is defined as the transcription and/or translation of a particular nucleotide sequence.

“Fragment” as the term is used herein, is a nucleic acid sequence that differs in length (i.e., in the number of nucleotides) from the length of a reference nucleic acid sequence, but retains essential properties of the reference molecule. Preferably, the fragment is at least about 50% of the length of the reference nucleic acid sequence. More preferably, the fragment is at least about 75% of the length of the reference nucleic acid sequence. Even more preferably, the fragment is at least about 95% of the length of the reference nucleic acid sequence.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic animals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked.

A “genome” is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

As used herein, “hybridization,” “hybridize(s)” or “capable of hybridizing” is understood to mean the forming of a double or triple stranded molecule or a molecule with partial double or triple stranded nature. Complementary sequences in the nucleic acids pair with each other to form a double helix. The resulting double-stranded nucleic acid is a “hybrid.” Hybridization may be between, for example two complementary or partially complementary sequences. The hybrid may have double-stranded regions and single stranded regions. The hybrid may be, for example, DNA:DNA, RNA:DNA or DNA:RNA. Hybrids may also be formed between modified nucleic acids (e.g., LNA compounds). One or both of the nucleic acids may be immobilized on a solid support. Hybridization techniques may be used to detect and isolate specific sequences, measure homology, or define other characteristics of one or both strands. The stability of a hybrid depends on a variety of factors including the length of complementarity, the presence of mismatches within the complementary region, the temperature and the concentration of salt in the reaction or nucleotide modifications in one of the two strands of the hybrid. Hybridizations are usually performed under stringent conditions, for example, at a salt concentration of no more than 1 M and a temperature of at least 25° C. For example, conditions of 5X SSPE (750 mM NaCl, 50 mM Na Phosphate, 5 mM EDTA, pH 7.4) or 100 mM MES, 1 M Na, 20 mM EDTA, 0.01% Tween-20 and a temperature of 25-50° C. are suitable for probe hybridizations. In a particularly preferred embodiment, hybridizations are performed at 40-50° C. Acetylated BSA and herring sperm DNA may be added to hybridization reactions. Hybridization conditions suitable for microarrays are described in the Gene Expression Technical Manual and the GeneChip Mapping Assay Manual available from Affymetrix (Santa Clara, Calif.).

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

As used herein, an “instructional material” includes a publication, a recording, a diagram, or any other medium of expression which can be used to communicate the usefulness of a compound, composition, vector, method or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence

As used herein, “microRNA” or “miRNA” describes small non-coding RNA molecules, generally about 15 to about 50 nucleotides in length, preferably 17-23 nucleotides, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at www.mirbase.org, incorporated by reference herein in its entirety for all purposes.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant,” as used herein, refers to either a nucleic acid or protein comprising a mutation.

“Naturally occurring” as used herein describes a composition that can be found in nature as distinct from being artificially produced. For example, a nucleotide sequence present in an organism, which can be isolated from a source in nature and which has not been intentionally modified by a person, is naturally occurring.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

As used herein, “polynucleotide” includes cDNA, RNA, DNA/RNA hybrid, anti-sense RNA, siRNA, miRNA, snoRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “recombinant DNA” as used herein is defined as DNA produced by joining pieces of DNA from different sources.

The term “recombinant polypeptide” as used herein is defined as a polypeptide produced by using recombinant DNA methods.

“Sample” or “biological sample” as used herein means a biological material from a subject, including but is not limited to organ, tissue, exosome, blood, plasma, saliva, urine and other body fluid. A sample can be any source of material obtained from a subject.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

“Synthetic mutant” includes any purposefully generated mutant or variant protein or nucleic acid. Such mutants can be generated by, for example, chemical mutagenesis, polymerase chain reaction (PCR) based approaches, or primer-based mutagenesis strategies well known to those skilled in the art.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by the invention include, but are not restricted to, oligonucleotides, nucleic acids, antibodies, cell membrane receptors, monoclonal antibodies and antisera reactive with specific antigenic determinants (such as on viruses, cells or other materials), drugs, peptides, cofactors, lectins, sugars, polysaccharides, cells, cellular membranes, and organelles. Targets are sometimes referred to in the art as anti-probes.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to the discovery that the level of expression or expression pattern of particular biomarkers is associated with bacterial infection. In some embodiments, one or more biomarkers associated with bacterial infection is up-regulated, or expressed at a higher than normal level. In other embodiments, one or more biomarkers associated with bacterial infection is down-regulated, or expressed at a lower than normal level. Thus, the invention relates to compositions and methods useful for the diagnosis, assessment, and characterization of bacterial infection in a subject in need thereof, based upon the expression level or expression pattern of one or more biomarkers that is associated with bacterial infection.

In various embodiments, the methods of the invention relate to methods of treating a bacterial infection in a subject, where the subject has been identified as having a differentially expressed level of one or more biomarkers associated with a bacterial infection, is administered an antibiotic. In some embodiments, the methods of the invention relate to altering a treatment of an infection in a subject who has been identified as not having a differentially expressed level of one or more biomarkers associated with a bacterial infection by discontinuing administration of an antibiotic.

In some embodiments, the infection is a respiratory infection. In a specific embodiment, the infection is bronchitis, pneumonia, chronic obstructive pulmonary disease (COPD), asthma, influenza, viral syndrome or sinusitis. In various embodiments of the compositions and methods of the invention described herein, biomarker associated with bacterial infection is one or more biomarkers set forth in Table 1. In other embodiments, biomarker associated with bacterial infection is at least one of ICAM1, ITGAL, ITGB2, PECAM1, FADS2, PLA2GA4, CTSG, IGFBP2, IGFBP6, MMP2, and ACOX3, or any combination thereof

### Assays and Methods of Diagnosis

In some embodiments, the invention relates to a screening assay of a subject to determine whether the subject has a differentially expressed level or pattern of one or more biomarkers one or more biomarkers listed in Table 1. In some embodiments, the invention relates to a screening assay of a subject to determine whether the subject has an elevated level of expression one or more biomarkers listed in Table 1. In other embodiments, the invention relates to a screening assay of a subject to determine whether the subject has a reduced level of expression of one or more biomarkers listed in Table 1. In one embodiment, the biomarker is a gene. The present invention provides methods of assessing the level of gene products, including mRNAs and polypeptides, in a subject. In various embodiments, the level of a gene product in the biological sample can be determined by assessing the amount of polypeptide present in the biological sample, the amount of mRNA present in the biological sample, the amount of activity of the gene product in the biological sample, the amount of binding activity of the gene product in the biological sample, or a combination thereof. In various embodiments, the gene product is a gene product of at least one of the biomarkers listed in Table 1. In one embodiment, the gene product is the gene product of at least one of ICAM1, ITGAL, ITGB2, PECAM1, FADS2, PLA2GA4, CTSG, IGFBP2, IGFBP6, MMP2, and ACOX3.

In one embodiment, the invention is a diagnostic assay for diagnosing a bacterial infection in a subject, by determining whether the subject has a differentially expressed level of one or more biomarkers listed in Table 1. In one embodiment, the invention is a diagnostic assay for diagnosing a bacterial infection in a subject, by determining whether the subject has an elevated level of expression one or more biomarkers listed in Table 1. In another embodiment, the invention is a diagnostic assay for diagnosing a bacterial infection in a subject, by determining whether the subject has a reduced level of expression of one or more biomarkers listed in Table 1. In yet another embodiment, the invention is a diagnostic assay for diagnosing a bacterial infection in a subject, by determining whether the subject has both a reduced level of expression of one or more biomarkers listed in Table 1 and an elevated level of expression one or more biomarkers listed in Table 1. In various embodiments, the level of a gene product in the biological sample can be determined by assessing the amount of polypeptide present in the biological sample, the amount of mRNA present in the biological sample, the amount of activity of the gene product in the biological sample, the amount of binding activity of the gene product in the biological sample, or a combination thereof. In various embodiments, the gene product is a gene product of one or more biomarkers listed in Table 1. In one embodiment, the gene product is the gene product of at least one of ICAM1, ITGAL, ITGB2, PECAM1, FADS2, PLA2GA4, CTSG, IGFBP2, IGFBP6, MMP2, and ACOX3.

In various embodiments, to determine whether the level of expression of one or more biomarkers listed in Table 1 is increased or reduced in a biological sample of the subject, the level of expression of the one or more biomarkers listed in Table 1 is compared with the level of at least one comparator control, such as a positive control, a negative control, a historical control, a historical norm, or the level of another reference molecule in the biological sample. The results of the diagnostic assay can be used alone, or in combination with other information from the subject, or other information from the biological sample obtained from the subject.

In some embodiments, the invention is a method of determining whether the subject has a differentially expressed level of one or more biomarkers listed in Table 1. In some embodiments, the invention is a method of determining whether the subject has an elevated level of expression one or more biomarkers listed in Table 1. In other embodiments, the invention is a method of determining whether a subject has a reduced level of expression of one or more biomarkers listed in Table 1. The present invention provides methods of assessing the level of gene products, including mRNAs and polypeptides, in a subject. In various embodiments, the level of a gene product in the biological sample can be determined by assessing the amount of polypeptide present in the biological sample, the amount of mRNA present in the biological sample, the amount of activity of the gene product in the biological sample, the amount of binding activity of the gene product in the biological sample, or a combination thereof. In various embodiments, the gene product is a gene product of one or more biomarkers listed in Table 1. In one embodiment, the gene product is the gene product of at least one of ICAM1, ITGAL, ITGB2, PECAM1, FADS2, PLA2GA4, CTSG, IGFBP2, IGFBP6, MMP2, and ACOX3. In some embodiments, the biological sample is a blood, plasma, saliva, or urine.

In one embodiment, the invention is a method of diagnosing a bacterial infection in a subject, by determining whether the subject has a differentially expressed level of one or more biomarkers listed in Table 1. In one embodiment, the invention is a method of diagnosing a bacterial infection in a subject, by determining whether the subject has an elevated level of expression one or more biomarkers listed in Table 1. In another embodiment, the invention is a method of diagnosing a bacterial infection in a subject, by determining whether the subject has a reduced level of expression of one or more biomarkers listed in Table 1. In various embodiments, the level of a gene product in the biological sample can be determined by assessing the amount of polypeptide present in the biological sample, the amount of mRNA present in the biological sample, the amount of activity of the gene product in the biological sample, the amount of binding activity of the gene product in the biological sample, or a combination thereof. In various embodiments, the gene product is a gene product of one or more biomarkers listed in Table 1. In one embodiment, the gene product is the gene product of at least one of ICAM1, ITGAL, ITGB2, PECAM1, FADS2, PLA2GA4, CTSG, IGFBP2, IGFBP6, MMP2, and ACOX3.

In various embodiments, to determine whether the level of expression of one or more biomarkers listed in Table 1 is increased or reduced in a biological sample of the subject, the level of expression of the at least one gene listed in Table 1 is compared with the level of at least one comparator control, such as a positive control, a negative control, a historical control, a historical norm, or the level of another reference molecule in the biological sample. The results of the diagnostic assay can be used alone, or in combination with other information from the subject, or other information from the biological sample obtained from the subject.

In various embodiments of the assays of the invention, the level of expression of the biomarker is determined to be elevated or increased when the level of expression of the biomarker is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator control.

In various embodiments of the methods of the invention, the level of expression of the biomarker is determined to be elevated or increased when the level of expression of the biomarker in the biological sample is increased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator.

In other various embodiments of the assays of the invention, the level of expression of the biomarker is determined to be reduced or decreased when the level of expression of the biomarker is reduced or decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator control.

In other various embodiments of the assays of the invention, the level of expression of the biomarker is determined to be reduced or decreased when the level of expression of the biomarker is reduced or decreased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator.

In one embodiment, the method comprises using a multi-dimensional non-linear algorithm to determine if the expression level of a set of biomarkers in the biological sample is statistically different than the expression level in a control sample. In various embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

In the assay methods of the invention, a test biological sample from a subject is assessed for the level of expression of one or more biomarkers listed in Table 1 in the biological sample obtained from the patient. The level of expression of the biomarker in the biological sample can be determined by assessing the amount of polypeptide gene product of the gene in the biological sample, the amount of mRNA gene product of the gene in the biological sample, the amount of activity of the gene product in the biological sample, the amount of binding activity of the gene product in the biological sample, or a combination thereof.

In various embodiments, the subject is a human subject, and may be of any race, sex and age. Representative subjects include those who are suspected of having a bacterial infection, those who have been diagnosed with respiratory infection or a bacterial infection, those who have developed respiratory infection, or those who at risk of developing respiratory infection or a bacterial infection. In some embodiments, the subject is suspected of having, or has been diagnosed as having, bronchitis, pneumonia, exacerbation of chronic obstructive pulmonary disease (COPD), exacerbation of asthma, influenza, viral syndrome, upper respiratory tract infection, common cold, otitis media, or sinusitis.

In various embodiments, the test sample is a biological sample (e.g., fluid, tissue, cell, cellular component, etc.) of the subject containing at least a fragment of a gene product (e.g., polypeptide or mRNA) of one or more biomarkers listed in Table 1. The biological sample can be a sample from any source which contains a polypeptide or a nucleic acid, such as a fluid, tissue, cell, cellular component, or a combination thereof. A biological sample can be obtained by appropriate methods, such as, by way of examples, blood draw, fluid draw, or biopsy. A biological sample can be used as the test sample; alternatively, a biological sample can be processed to enhance access to the polypeptides or nucleic acids, or copies of the nucleic acids, and the processed biological sample can then be used as the test sample. For example, in various embodiments, nucleic acid (e.g., mRNA, cDNA prepared from mRNA, etc.) is prepared from a biological sample, for use in the assays and methods. Alternatively or in addition, if desired, an amplification method can be used to amplify nucleic acids comprising all or a fragment of an mRNA in a biological sample, for use as the test sample in the assessment of the level in the biological sample. In some embodiments, the biological sample is blood, plasma, saliva, or urine. In another embodiment, the biological sample is blood. In one embodiment, the biological sample comprises a PBMC.

In various embodiments of the invention, methods of measuring polypeptide levels in a biological sample obtained from a patient include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

Methods useful for the detection, identification and measurement of nucleic acids in the methods of the invention include high throughput RNA and DNA sequencing.

In some embodiments, quantitative hybridization methods, such as Southern analysis, Northern analysis, or in situ hybridizations, can be used (see Current Protocols in Molecular Biology, Ausubel, F. et al., eds., John Wiley & Sons, including all supplements). A “nucleic acid probe,” as used herein, can be a DNA probe or an RNA probe. The probe can be, for example, a gene, a gene fragment (e.g., one or more exons), a vector comprising the gene, a probe or primer, etc. For representative examples of use of nucleic acid probes, see, for example, U.S. Pat. Nos. 5,288,611 and 4,851,330. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target mRNA or cDNA. The hybridization sample is maintained under conditions which are sufficient to allow specific hybridization of the nucleic acid probe to mRNA or cDNA. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe having a mRNA or cDNA in the test sample, the level of the mRNA or cDNA in the sample can be assessed. More than one nucleic acid probe can also be used concurrently in this method. Specific hybridization of any one of the nucleic acid probes is indicative of the presence of the mRNA or cDNA of interest, as described herein.

Alternatively, a peptide nucleic acid (PNA) probe can be used instead of a nucleic acid probe in the quantitative hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a target nucleic acid sequence. Hybridization of the PNA probe to a nucleic acid sequence is used to determine the level of the target nucleic acid in the biological sample.

In another embodiment, arrays of oligonucleotide probes that are complementary to target nucleic acid sequences in the biological sample obtained from a subject can be used to determine the level a nucleic acid in the biological sample obtained from a subject. The array of oligonucleotide probes can be used to determine the level of target nucleic alone, or the level of the target nucleic in relation to the level of one or more other nucleic acids in the biological sample. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and its level is quantified. Hybridization and quantification are generally carried out by methods described herein and also in, e.g., published PCT Application Nos. WO 92/10092 and WO 95/11995, and U.S. Pat. No. 5,424,186. In brief, a target nucleic acid sequence is amplified by well-known amplification techniques, e.g., PCR. Typically, this involves the use of primer sequences that are complementary to the target nucleic acid. Asymmetric PCR techniques may also be used. Amplified target, generally incorporating a label, is then hybridized with the array under appropriate conditions. Upon completion of hybridization and washing of the array, the array is scanned to determine the quantity of hybridized nucleic acid. The hybridization data obtained from the scan is typically in the form of fluorescence intensities as a function of quantity, or relative quantity, of the target nucleic acid in the biological sample. The target nucleic acid can be hybridized to the array in combination with one or more comparator controls (e.g., positive control, negative control, quantity control, etc.) to improve quantification of the target nucleic acid in the sample.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of 32P, 33P, 35S or 3H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from the cells using known techniques. Nucleic acid herein refers to RNA, including mRNA, and DNA, including cDNA. The nucleic acid can be double-stranded or single-stranded (i.e., a sense or an antisense single strand) and can be complementary to a nucleic acid encoding a polypeptide. The nucleic acid content may also be an RNA or DNA extraction performed on a biological sample, including a biological fluid and fresh or fixed tissue sample.

There are many methods known in the art for the detection and quantification of specific nucleic acid sequences and new methods are continually reported. A great majority of the known specific nucleic acid detection and quantification methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope, a fluorescent dye or the other well-known materials. Another type of process for the specific detection of nucleic acids in a biological sample known in the art are the hybridization methods as exemplified by U.S. Pat. Nos. 6,159,693 and 6,270,974, and related patents. To briefly summarize one of those methods, a nucleic acid probe of at least 10 nucleotides, preferably at least 15 nucleotides, more preferably at least 25 nucleotides, having a sequence complementary to a nucleic acid of interest is hybridized in a sample, subjected to depolymerizing conditions, and the sample is treated with an ATP/luciferase system, which will luminesce if the nucleic sequence is present. In quantitative Southern blotting, the level of the nucleic acid of interest can be compared with the level of a second nucleic acid of interest, and/or to one or more comparator control nucleic acids (e.g., positive control, negative control, quantity control, etc.).

Many methods useful for the detection and quantification of nucleic acid takes advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed elsewhere herein. Most preferably the detection of the duplex is done using at least one primer directed to the nucleic acid of interest. In yet another embodiment of PCR, the detection of the hybridized duplex comprises electrophoretic gel separation followed by dye-based visualization.

Typical hybridization and washing stringency conditions depend in part on the size (i.e., number of nucleotides in length) of the oligonucleotide probe, the base composition and monovalent and divalent cation concentrations (Ausubel et al., 1994, eds Current Protocols in Molecular Biology).

In a preferred embodiment, the process for determining the quantitative and qualitative profile of the nucleic acid of interest according to the present invention is characterized in that the amplifications are real-time amplifications performed using a labeled probe, preferably a labeled hydrolysis-probe, capable of specifically hybridizing in stringent conditions with a segment of the nucleic acid of interest. The labeled probe is capable of emitting a detectable signal every time each amplification cycle occurs, allowing the signal obtained for each cycle to be measured.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 65° C. to 75° C. Preferably, the Tm for any one of the hydrolysis-probes of the present invention is in the range of about 67° C. to about 70° C. Most preferably, the Tm applied for any one of the hydrolysis-probes of the present invention is about 67° C.

In one aspect, the invention includes a primer that is complementary to a nucleic acid of interest, and more particularly the primer includes 12 or more contiguous nucleotides substantially complementary to the nucleic acid of interest. Preferably, a primer featured in the invention includes a nucleotide sequence sufficiently complementary to hybridize to a nucleic acid sequence of about 12 to 25 nucleotides. More preferably, the primer differs by no more than 1, 2, or 3 nucleotides from the target flanking nucleotide sequence In another aspect, the length of the primer can vary in length, preferably about 15 to 28 nucleotides in length (e.g., 16, 17, 18, 19, 20, 21, 22, 23, 24, 25, 26, or 27 nucleotides in length).

### Treatments

In one aspect, the invention provides a method of treating a respiratory infection in a subject. For example, in certain embodiments, the subject has been identified as having a differentially expressed level of one or more biomarkers listed in Table 1. In one embodiment, the method comprises administering to the subject an effective amount of an antibiotic.

In one embodiment, the respiratory infection is a lower respiratory infection. In another embodiment, the respiratory infection is a lower respiratory tract infection. In various embodiments, the respiratory infection is bronchitis, pneumonia, exacerbation of chronic obstructive pulmonary disease (COPD), exacerbation of asthma, influenza, viral syndrome or sinusitis.

In one embodiment, the method comprises administering to the subject an effective amount of an antibiotic. In some embodiments, the antibiotic includes Cefradine (cephradine), cefrotil, cefroxadine, cefsumide, ceftaroline, ceftazidime, cefteram, ceftezole, ceftibuten, ceftiofur, ceftiolene, ceftioxide, ceftizoxime, Ceftobiprole, ceftriaxone, cefuracetime, cefuroxime, cefuzonam, cephalosporin, chloramphenicol, cilastatin, ciprofloxacin, clarithromycin, clinafloxacin, clindamycin, cloxacillin, demeclocycline, dicloxacillin, dirithromycin, doripenem, doxycycline, enoxacin, ertapenem, erythromycin, flucloxacillin, flumequine, fluoroquinolone, gatifloxacin, gemifloxacin, gentamicin, grepafloxacin, imipenem, kanamycin, ketolide, levofloxacin, Lincomycin, linezolid, Linezolid., lomefloxacin, meropenem, metronidazole, mezlocillin, minocycline, moxifloxacin, mycobutin, nadifloxacin, nafcillin, nalidixic acid, neomycin, netilmicin, nitrofurantoin, norfloxacin, ofloxacin, oxacillin, oxolinic acid, oxytetracycline, paromomycin, pazufloxacin, pefloxacin, penicillin g, penicillin v, piperacillin, piromidic acid pipemidic acid, pivampicillin, pivmecillinam, primaxin, prulifloxacin, rifampin, rosoxacin, roxithromycin, rufloxacin, sitafloxacin, sparfloxacin, streptomycin, sulfamethizole, sulfamethoxazole, sulfisoxazole, Teicoplanin, Telavancin, telithromycin, temafloxacin, tetracycline, ticarcillin, tobramycin, tosufloxacin, trimethoprim-sulfamethoxazole, trovafloxacin, vancocin, vancomycin, and lipopeptide.

In another embodiment, the antibiotic is selected from Amoxicillin, Ampicillin, Cloxacillin, Dicloxacillin, Nafcillin, Oxacillin, Penicillin G, Penicillin V, Piperacillin, Cefadroxil (cefadroxyl), Cefalexin (cephalexin), Cefalotin (cephalothin), Cefapirin (cephapirin), Cefazolin (cephazolin), Cefradine (cephradine), Cefaclor, Cefotetan, Cefoxitin, Cefprozil (cefproxil), Cefuroxime, Cefdinir, Cefixime, Cefotaxime, Cefpodoxime, Ceftizoxime, Ceftriaxone, Ceftazidime, Cefepime, Ceftobiprole, Ceftaroline, Aztreonam, Imipenem, Imipenem, cilastatin, Doripenem, Meropenem, Ertapenem, Azithromycin, Erythromycin, Clarithromycin, Dirithromycin, Roxithromycin, Clindamycin, Lincomycin, Amikacin, Gentamicin, Tobramycin, Ciprofloxacin, Levofloxacin, Moxifloxacin, Trimethoprim-Sulfamethoxazole, Doxycycline, Tetracycline, Vancomycin, Teicoplanin, Telavancin, and Linezolid.

In some embodiments, the method further comprises administering to the subject an effective amount of a second therapeutic. In one embodiment, the second therapeutic is an antibiotic or an antiviral agent.

Exemplary antiviral agents that can be used with the methods of the invention include, but are not limited to, Aciclovir, Acyclovir, Adefovir, Amantadine, Amprenavir, Ampligen, Arbidol, Atazanavir, Atripla, Balavir, Cidofovir, Combivir, Darunavir, Docosanol, Edoxudine, Entecavir, Ecoliever, Famciclovir, Fomivirsen, Foscarnet, Fosfonet, Ganciclovir, Ibacitabine, Imunovir, Idoxuridine, Imiquimod, Inosine, Interferon type III, Interferon type II, Interferon type I, Interferon, Lopinavir, Loviride, Moroxydine, Methisazone, Nexavir, Nitazoxanide, Novir, Peginterferon alfa-2a, Penciclovir, Peramivir, Pleconaril, Podophyllotoxin, Ribavirin, Rimantadine, Pyramidine, Sofosbuvir, Telaprevir, Trifluridine, Trizivir, Tromantadine, Truvada, Valaciclovir, Valganciclovir, Vicriviroc, Vidarabine, Viramidine, Zalcitabine, Zanamivir.

Additionally, therapeutic agents suitable for administration to a particular subject can be identified by detecting one or more biomarkers listed in Table 1. Accordingly, treatments or therapeutic regimens for use in subjects having a bacterial infection can be selected based on the level of one or more biomarkers listed in Table 1 and compared to a reference value. In various embodiments, a recommendation is made on whether to initiate or continue administration of an antibiotic. In some embodiments, a recommendation is made on whether to discontinue administration of an antibiotic.

Any drug or combination of drugs disclosed herein may be administered to a subject to treat a disease. The drugs herein can be formulated in any number of ways, often according to various known formulations in the art or as disclosed or referenced herein.

In various embodiments, any drug or combination of drugs disclosed herein is not administered to a subject to treat a disease or infection. In these embodiments, the practitioner may refrain from administering the drug or combination of drugs, may recommend that the subject not be administered the drug or combination of drugs or may prevent the subject from being administered the drug or combination of drugs.

In various embodiments, one or more additional drugs may be optionally administered in addition to those that are recommended or have been administered. An additional drug will typically not be any drug that is not recommended or that should be avoided.

### Kits

The present invention also pertains to kits useful in the methods of the invention. Such kits comprise various combinations of components useful in any of the methods described elsewhere herein, including for example, materials for quantitatively analyzing a biomarker of the invention (e.g., polypeptide and/or nucleic acid), materials for assessing the activity of a biomarker of the invention (e.g., polypeptide and/or nucleic acid), and instructional material. For example, in one embodiment, the kit comprises components useful for the quantification of a desired nucleic acid in a biological sample. In another embodiment, the kit comprises components useful for the quantification of a desired polypeptide in a biological sample. In a further embodiment, the kit comprises components useful for the assessment of the activity (e.g., enzymatic activity, substrate binding activity, etc.) of a desired polypeptide in a biological sample.

In one embodiment, the kit comprises a reagent for measuring the level of one or more biomarkers in a biological sample of a subject. For example, in one embodiment, the one or more biomarkers are selected from Table 1. In another embodiment, the one or more biomarkers are selected from ICAM1, ITGAL, ITGB2, PECAM1, FADS2, PLA2GA4, CTSG, IGFBP2, IGFBP6, MMP2, and ACOX3. In some embodiments, the biological sample is blood, urine, saliva or plasma. In one embodiment, the biological sample comprises a PBMC.

In a further embodiment, the kit comprises the components of an assay for selecting a treatment to be administered to a subject in need thereof, containing instructional material and the components for determining whether the level of a biomarker of the invention in a biological sample obtained from the subject is modulated during or after administration of the treatment. In various embodiments, to determine whether the level of a biomarker of the invention is modulated in a biological sample obtained from the subject, the level of the biomarker is compared with the level of at least one comparator control contained in the kit, such as a positive control, a negative control, a historical control, a historical norm, or the level of another reference molecule in the biological sample. In certain embodiments, the ratio of the biomarker and a reference molecule is determined to aid in the monitoring of the treatment.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Identification of Bacterial Biomarkers

The materials and methods employed in these experiments are now described.

213 hospitalized subjects >18 years of age consented to participate in the study. Clinical data and peripheral blood RNA was collected, and comprehensive microbiologic testing was performed. Using stringent criteria 98 subjects could be classified as: absence of bacterial infection, N=55 (viral infection alone) or presence of bacterial infection, N=43 (28 bacterial alone and 15 mixed viral-bacterial infection). Gene expression analysis was performed using RNASeq and quantitative real-time PCR (qPCR). Univariate gene selection and screening was based on the Bonferroni-corrected Wilcoxon test. Constrained logistic models to predict bacterial infection were fit using screened LASSO and supervised principal components analysis. Cross-validated (CV) AUC was used to select the screening level and LASSO penalty, and the entire procedure was nested within an outer CV loop to estimate the AUC of the adaptive procedures.

RNAseq analysis identified 249 genes differentially expressed in LRTI subjects with bacterial infection, whether viral co-infection was present or absent (Table 1). These genes implicated the involvement of obvious infection-related (e.g. viral infection, viral replication), as well as novel (e.g. cell phagocytosis, neuromuscular disease), pathways. Interestingly, subjects with co-infection (bacterial plus viral) displayed a transcriptomic profile more similar to those with a bacterial infection alone. Importantly, while selected clinical variables (BUN, WBC, infiltrates, congestion) were capable of discriminating between bacterial and non-bacterial LRTI with surprisingly high accuracy (AUC=0.81), gene expression patterns performed as well as clinical variables.

TABLE 1

| | Wilcoxon | Mean | Mean | Log Fold | Fold |
|---|---|---|---|---|---|
| Gene Name | p-value | Bacterial | Viral | Change | Change |
| PCOLCE2 | 1.2E−04 | 5.8E+00 | 3.6E+00 | 2.1E+00 | 4.4E+00 |
| TNFAIP8L3 | 1.2E−04 | 4.5E+00 | 2.5E+00 | 2.0E+00 | 4.1E+00 |
| ZDHHC19 | 3.3E−04 | 7.5E+00 | 5.8E+00 | 1.7E+00 | 3.3E+00 |
| FSTL4 | 6.3E−05 | 5.0E+00 | 3.4E+00 | 1.6E+00 | 3.0E+00 |
| AP3B2 | 4.3E−04 | 6.3E+00 | 4.7E+00 | 1.5E+00 | 2.9E+00 |
| S100A12 | 2.2E−04 | 1.4E+01 | 1.3E+01 | 1.3E+00 | 2.5E+00 |
| GPR84 | 2.1E−04 | 8.5E+00 | 7.2E+00 | 1.3E+00 | 2.5E+00 |
| KAL1 | 4.3E−04 | 4.9E+00 | 3.7E+00 | 1.2E+00 | 2.4E+00 |
| C11orf82 | 1.1E−04 | 8.2E+00 | 7.0E+00 | 1.2E+00 | 2.3E+00 |
| SIGLEC10 | 6.3E−05 | 1.2E+01 | 1.1E+01 | 1.2E+00 | 2.3E+00 |
| LTA4H | 1.8E−04 | 1.4E+01 | 1.3E+01 | 1.1E+00 | 2.1E+00 |
| FAM101B | 6.3E−05 | 1.2E+01 | 1.1E+01 | 1.0E+00 | 2.0E+00 |
| TSPO | 4.9E−04 | 1.3E+01 | 1.2E+01 | 8.8E−01 | 1.8E+00 |
| ANPEP | 6.3E−05 | 1.4E+01 | 1.3E+01 | 8.5E−01 | 1.8E+00 |
| TMEM169 | 1.9E−04 | 7.2E+00 | 6.3E+00 | 8.2E−01 | 1.8E+00 |
| CAT | 1.8E−04 | 1.3E+01 | 1.2E+01 | 8.0E−01 | 1.7E+00 |
| WIPI1 | 1.5E−04 | 1.0E+01 | 9.3E+00 | 7.9E−01 | 1.7E+00 |
| CMTM4 | 3.8E−04 | 7.9E+00 | 7.1E+00 | 7.9E−01 | 1.7E+00 |
| ACER3 | 3.8E−04 | 9.6E+00 | 8.8E+00 | 7.9E−01 | 1.7E+00 |
| FAM173B | 2.4E−04 | 6.9E+00 | 6.1E+00 | 7.8E−01 | 1.7E+00 |
| PGLYRP1 | 3.2E−04 | 1.0E+01 | 9.5E+00 | 7.5E−01 | 1.7E+00 |
| IER3 | 2.8E−04 | 9.2E+00 | 8.4E+00 | 7.4E−01 | 1.7E+00 |
| EEPD1 | 1.1E−04 | 9.4E+00 | 8.7E+00 | 7.3E−01 | 1.7E+00 |
| ERP27 | 2.9E−04 | 6.8E+00 | 6.1E+00 | 6.9E−01 | 1.6E+00 |
| TKT | 9.8E−05 | 1.5E+01 | 1.4E+01 | 6.8E−01 | 1.6E+00 |
| GAPDH | 6.3E−05 | 1.6E+01 | 1.5E+01 | 6.8E−01 | 1.6E+00 |
| F8 | 2.9E−04 | 7.7E+00 | 7.0E+00 | 6.8E−01 | 1.6E+00 |
| AMPD3 | 2.2E−04 | 1.1E+01 | 1.0E+01 | 6.6E−01 | 1.6E+00 |
| PGCP | 2.2E−04 | 1.2E+01 | 1.1E+01 | 6.6E−01 | 1.6E+00 |
| TBXAS1 | 1.5E−04 | 1.3E+01 | 1.2E+01 | 6.5E−01 | 1.6E+00 |
| PTAFR | 5.0E−04 | 1.4E+01 | 1.3E+01 | 6.5E−01 | 1.6E+00 |
| SFXN5 | 4.5E−04 | 9.6E+00 | 9.0E+00 | 6.5E−01 | 1.6E+00 |
| PPM1M | 2.2E−04 | 1.2E+01 | 1.2E+01 | 6.4E−01 | 1.6E+00 |
| VIM | 2.1E−04 | 1.6E+01 | 1.5E+01 | 6.3E−01 | 1.6E+00 |
| ABHD2 | 2.4E−04 | 1.4E+01 | 1.3E+01 | 6.3E−01 | 1.5E+00 |
| ACPP | 2.2E−04 | 9.8E+00 | 9.2E+00 | 6.2E−01 | 1.5E+00 |
| VWA5A | 5.0E−04 | 8.6E+00 | 8.0E+00 | 6.2E−01 | 1.5E+00 |
| ITGAM | 3.0E−04 | 1.5E+01 | 1.4E+01 | 6.2E−01 | 1.5E+00 |
| CEL | 5.0E−04 | 4.0E+00 | 3.4E+00 | 6.1E−01 | 1.5E+00 |
| PHKA2 | 3.9E−04 | 1.1E+01 | 1.0E+01 | 6.1E−01 | 1.5E+00 |
| GLT25D1 | 4.5E−04 | 1.2E+01 | 1.2E+01 | 6.1E−01 | 1.5E+00 |
| ANO10 | 4.3E−04 | 9.7E+00 | 9.1E+00 | 6.1E−01 | 1.5E+00 |
| FAM114A1 | 1.2E−04 | 6.7E+00 | 6.1E+00 | 6.1E−01 | 1.5E+00 |
| GSN | 2.2E−04 | 1.3E+01 | 1.2E+01 | 6.0E−01 | 1.5E+00 |
| C19orf38 | 1.5E−04 | 1.2E+01 | 1.1E+01 | 6.0E−01 | 1.5E+00 |
| ITGB2 | 1.2E−04 | 1.6E+01 | 1.6E+01 | 5.8E−01 | 1.5E+00 |
| PRCP | 3.3E−04 | 1.2E+01 | 1.2E+01 | 5.8E−01 | 1.5E+00 |
| PECAM1 | 2.2E−04 | 1.3E+01 | 1.3E+01 | 5.8E−01 | 1.5E+00 |
| LOC100129034 | 1.8E−04 | 1.2E+01 | 1.2E+01 | 5.8E−01 | 1.5E+00 |
| SPG21 | 7.4E−05 | 1.2E+01 | 1.1E+01 | 5.7E−01 | 1.5E+00 |
| NMNAT1 | 1.7E−04 | 8.7E+00 | 8.2E+00 | 5.6E−01 | 1.5E+00 |
| AP3S2 | 4.5E−04 | 6.5E+00 | 5.9E+00 | 5.6E−01 | 1.5E+00 |
| CD99L2 | 6.3E−05 | 1.0E+01 | 9.5E+00 | 5.5E−01 | 1.5E+00 |
| RAB3A | 6.3E−05 | 5.7E+00 | 5.2E+00 | 5.5E−01 | 1.5E+00 |
| HYAL2 | 2.8E−04 | 8.6E+00 | 8.0E+00 | 5.5E−01 | 1.5E+00 |
| PGK1 | 3.2E−04 | 1.4E+01 | 1.3E+01 | 5.5E−01 | 1.5E+00 |
| SYTL1 | 4.7E−04 | 1.1E+01 | 1.0E+01 | 5.5E−01 | 1.5E+00 |
| DHCR7 | 7.9E−05 | 7.9E+00 | 7.3E+00 | 5.5E−01 | 1.5E+00 |
| GLTP | 1.2E−04 | 1.1E+01 | 1.0E+01 | 5.4E−01 | 1.5E+00 |
| CLTC | 2.0E−04 | 1.3E+01 | 1.3E+01 | 5.3E−01 | 1.4E+00 |
| CHP | 1.1E−04 | 1.2E+01 | 1.1E+01 | 5.3E−01 | 1.4E+00 |
| PIK3CB | 2.0E−04 | 1.0E+01 | 9.8E+00 | 5.3E−01 | 1.4E+00 |
| GALNT2 | 3.7E−04 | 1.1E+01 | 1.1E+01 | 5.3E−01 | 1.4E+00 |
| BICD2 | 4.3E−04 | 1.2E+01 | 1.1E+01 | 5.2E−01 | 1.4E+00 |
| DUS2L | 7.1E−05 | 9.1E+00 | 8.6E+00 | 5.2E−01 | 1.4E+00 |
| NEK6 | 4.0E−04 | 1.0E+01 | 9.8E+00 | 5.2E−01 | 1.4E+00 |
| CCNY | 4.5E−04 | 1.2E+01 | 1.2E+01 | 5.1E−01 | 1.4E+00 |
| NACC2 | 4.3E−04 | 1.1E+01 | 1.1E+01 | 5.1E−01 | 1.4E+00 |
| ACAD8 | 9.8E−05 | 9.0E+00 | 8.4E+00 | 5.1E−01 | 1.4E+00 |
| ZFP106 | 4.5E−04 | 1.3E+01 | 1.2E+01 | 5.0E−01 | 1.4E+00 |
| ERICH1 | 2.4E−04 | 9.6E+00 | 9.1E+00 | 5.0E−01 | 1.4E+00 |
| OXSR1 | 1.3E−04 | 1.1E+01 | 1.1E+01 | 5.0E−01 | 1.4E+00 |
| PLEKHM3 | 1.3E−04 | 9.7E+00 | 9.2E+00 | 5.0E−01 | 1.4E+00 |
| VAT1 | 6.3E−05 | 1.0E+01 | 9.7E+00 | 5.0E−01 | 1.4E+00 |
| SDHC | 6.3E−05 | 1.1E+01 | 1.0E+01 | 4.9E−01 | 1.4E+00 |
| FXR1 | 6.3E−05 | 1.0E+01 | 1.0E+01 | 4.9E−01 | 1.4E+00 |
| PLD2 | 1.6E−04 | 8.8E+00 | 8.3E+00 | 4.9E−01 | 1.4E+00 |
| ARPC1B | 2.2E−04 | 1.4E+01 | 1.4E+01 | 4.8E−01 | 1.4E+00 |
| FLII | 1.4E−04 | 1.3E+01 | 1.3E+01 | 4.8E−01 | 1.4E+00 |
| PMM1 | 2.3E−04 | 8.4E+00 | 7.9E+00 | 4.8E−01 | 1.4E+00 |
| ZDHHC7 | 1.4E−04 | 1.2E+01 | 1.1E+01 | 4.7E−01 | 1.4E+00 |
| PRDX5 | 4.7E−04 | 1.2E+01 | 1.1E+01 | 4.7E−01 | 1.4E+00 |
| ACADVL | 2.5E−04 | 1.2E+01 | 1.1E+01 | 4.7E−01 | 1.4E+00 |
| ENO1 | 2.2E−04 | 1.4E+01 | 1.4E+01 | 4.7E−01 | 1.4E+00 |
| GSR | 2.9E−04 | 1.1E+01 | 1.1E+01 | 4.7E−01 | 1.4E+00 |
| BBS1 | 1.4E−04 | 7.6E+00 | 7.1E+00 | 4.7E−01 | 1.4E+00 |
| TUBGCP3 | 6.3E−05 | 9.9E+00 | 9.5E+00 | 4.7E−01 | 1.4E+00 |
| CERK | 3.7E−04 | 1.1E+01 | 1.1E+01 | 4.7E−01 | 1.4E+00 |
| MTF1 | 4.9E−04 | 1.2E+01 | 1.1E+01 | 4.6E−01 | 1.4E+00 |
| CIDEB | 3.7E−04 | 1.1E+01 | 1.0E+01 | 4.6E−01 | 1.4E+00 |
| ING1 | 1.6E−04 | 9.5E+00 | 9.0E+00 | 4.6E−01 | 1.4E+00 |
| NDRG3 | 6.3E−05 | 1.0E+01 | 9.9E+00 | 4.5E−01 | 1.4E+00 |
| PSMD4 | 3.0E−04 | 1.1E+01 | 1.0E+01 | 4.5E−01 | 1.4E+00 |
| PELO | 4.3E−04 | 9.5E+00 | 9.0E+00 | 4.5E−01 | 1.4E+00 |
| GTF2F2 | 2.9E−04 | 8.9E+00 | 8.4E+00 | 4.5E−01 | 1.4E+00 |
| TESK2 | 4.4E−04 | 9.0E+00 | 8.6E+00 | 4.5E−01 | 1.4E+00 |
| VPS25 | 8.6E−05 | 9.6E+00 | 9.1E+00 | 4.4E−01 | 1.4E+00 |
| ACSS2 | 1.2E−04 | 1.0E+01 | 1.0E+01 | 4.4E−01 | 1.4E+00 |
| S100A6 | 3.0E−04 | 1.4E+01 | 1.4E+01 | 4.4E−01 | 1.4E+00 |
| PSMB4 | 6.3E−05 | 1.2E+01 | 1.1E+01 | 4.4E−01 | 1.4E+00 |
| PLCB2 | 1.8E−04 | 1.3E+01 | 1.3E+01 | 4.3E−01 | 1.4E+00 |
| RAB11FIP4 | 2.1E−04 | 1.1E+01 | 1.1E+01 | 4.3E−01 | 1.4E+00 |
| MACF1 | 4.3E−04 | 1.3E+01 | 1.2E+01 | 4.3E−01 | 1.3E+00 |
| ACTG1 | 6.3E−05 | 1.6E+01 | 1.6E+01 | 4.3E−01 | 1.3E+00 |
| CDC37 | 1.4E−04 | 1.2E+01 | 1.1E+01 | 4.3E−01 | 1.3E+00 |
| ALAD | 7.1E−05 | 1.0E+01 | 9.6E+00 | 4.3E−01 | 1.3E+00 |
| SGSH | 2.0E−04 | 9.7E+00 | 9.3E+00 | 4.2E−01 | 1.3E+00 |
| PSMD6 | 2.6E−04 | 1.0E+01 | 1.0E+01 | 4.2E−01 | 1.3E+00 |
| COG4 | 6.9E−05 | 1.0E+01 | 9.6E+00 | 4.2E−01 | 1.3E+00 |
| IRGQ | 6.3E−05 | 8.4E+00 | 8.0E+00 | 4.2E−01 | 1.3E+00 |
| FAM168B | 4.5E−04 | 1.1E+01 | 1.1E+01 | 4.2E−01 | 1.3E+00 |
| TRIM41 | 1.1E−04 | 9.8E+00 | 9.4E+00 | 4.2E−01 | 1.3E+00 |
| LAMP1 | 4.0E−04 | 1.3E+01 | 1.3E+01 | 4.1E−01 | 1.3E+00 |
| SETD8 | 2.3E−04 | 9.6E+00 | 9.2E+00 | 4.1E−01 | 1.3E+00 |
| NFKB1 | 2.0E−04 | 1.2E+01 | 1.2E+01 | 4.1E−01 | 1.3E+00 |
| WDR37 | 3.1E−04 | 1.1E+01 | 1.0E+01 | 4.1E−01 | 1.3E+00 |
| AGTRAP | 2.6E−04 | 1.2E+01 | 1.2E+01 | 4.1E−01 | 1.3E+00 |
| PRPF18 | 3.3E−04 | 9.2E+00 | 8.8E+00 | 4.0E−01 | 1.3E+00 |
| AKAP8L | 5.0E−04 | 1.0E+01 | 1.0E+01 | 4.0E−01 | 1.3E+00 |
| NADSYN1 | 3.2E−04 | 1.1E+01 | 1.0E+01 | 4.0E−01 | 1.3E+00 |
| TTC15 | 2.4E−04 | 9.7E+00 | 9.3E+00 | 4.0E−01 | 1.3E+00 |
| ATP5F1 | 4.3E−04 | 1.2E+01 | 1.1E+01 | 4.0E−01 | 1.3E+00 |
| UGGT1 | 1.6E−04 | 1.1E+01 | 1.1E+01 | 3.9E−01 | 1.3E+00 |
| EHD1 | 2.2E−04 | 1.3E+01 | 1.3E+01 | 3.9E−01 | 1.3E+00 |
| DAGLB | 9.8E−05 | 9.8E+00 | 9.4E+00 | 3.9E−01 | 1.3E+00 |
| SUCLG1 | 4.0E−04 | 1.0E+01 | 9.7E+00 | 3.9E−01 | 1.3E+00 |
| ALDH3B1 | 3.8E−04 | 1.1E+01 | 1.1E+01 | 3.9E−01 | 1.3E+00 |
| SETD3 | 1.4E−04 | 1.0E+01 | 1.0E+01 | 3.9E−01 | 1.3E+00 |
| ARHGEF6 | 1.3E−04 | 1.2E+01 | 1.2E+01 | 3.9E−01 | 1.3E+00 |
| POLR2B | 2.6E−04 | 1.1E+01 | 1.1E+01 | 3.8E−01 | 1.3E+00 |
| ANXA7 | 2.4E−04 | 1.2E+01 | 1.1E+01 | 3.8E−01 | 1.3E+00 |
| ZNF341 | 3.4E−04 | 8.3E+00 | 8.0E+00 | 3.8E−01 | 1.3E+00 |
| C17orf85 | 1.8E−04 | 9.7E+00 | 9.3E+00 | 3.8E−01 | 1.3E+00 |
| PGPEP1 | 6.3E−05 | 9.8E+00 | 9.5E+00 | 3.8E−01 | 1.3E+00 |
| MED22 | 1.2E−04 | 9.7E+00 | 9.4E+00 | 3.8E−01 | 1.3E+00 |
| LOC100507417 | 1.3E−04 | 1.0E+01 | 9.9E+00 | 3.8E−01 | 1.3E+00 |
| KCNK6 | 4.0E−04 | 8.5E+00 | 8.1E+00 | 3.8E−01 | 1.3E+00 |
| HADHA | 2.4E−04 | 1.2E+01 | 1.2E+01 | 3.8E−01 | 1.3E+00 |
| CSDE1 | 1.7E−04 | 1.4E+01 | 1.3E+01 | 3.7E−01 | 1.3E+00 |
| TGOLN2 | 2.0E−04 | 1.4E+01 | 1.3E+01 | 3.7E−01 | 1.3E+00 |
| CASP9 | 3.3E−04 | 9.2E+00 | 8.8E+00 | 3.7E−01 | 1.3E+00 |
| MYH9 | 2.8E−04 | 1.6E+01 | 1.6E+01 | 3.7E−01 | 1.3E+00 |
| PSMD1 | 3.1E−04 | 1.1E+01 | 1.0E+01 | 3.7E−01 | 1.3E+00 |
| PGAM1 | 2.3E−04 | 1.2E+01 | 1.2E+01 | 3.7E−01 | 1.3E+00 |
| GPR108 | 4.3E−04 | 1.1E+01 | 1.1E+01 | 3.6E−01 | 1.3E+00 |
| USP7 | 2.6E−04 | 1.2E+01 | 1.2E+01 | 3.6E−01 | 1.3E+00 |
| KLHL18 | 3.7E−04 | 9.9E+00 | 9.6E+00 | 3.6E−01 | 1.3E+00 |
| TBC1D2B | 2.4E−04 | 1.1E+01 | 1.1E+01 | 3.6E−01 | 1.3E+00 |
| GIT2 | 3.4E−04 | 1.2E+01 | 1.2E+01 | 3.6E−01 | 1.3E+00 |
| ELOVL1 | 9.8E−05 | 1.1E+01 | 1.1E+01 | 3.6E−01 | 1.3E+00 |
| ACTB | 1.6E−04 | 1.8E+01 | 1.7E+01 | 3.6E−01 | 1.3E+00 |
| SEC13 | 2.9E−04 | 1.0E+01 | 1.0E+01 | 3.5E−01 | 1.3E+00 |
| ACTR1A | 2.6E−04 | 1.2E+01 | 1.2E+01 | 3.5E−01 | 1.3E+00 |
| PSMC1 | 1.2E−04 | 1.1E+01 | 1.1E+01 | 3.5E−01 | 1.3E+00 |
| AUP1 | 3.9E−04 | 1.1E+01 | 1.1E+01 | 3.5E−01 | 1.3E+00 |
| PARK7 | 2.4E−04 | 1.1E+01 | 1.1E+01 | 3.5E−01 | 1.3E+00 |
| KIAA0922 | 7.4E−05 | 1.2E+01 | 1.1E+01 | 3.5E−01 | 1.3E+00 |
| RPN1 | 3.6E−04 | 1.3E+01 | 1.2E+01 | 3.5E−01 | 1.3E+00 |
| FDPS | 7.9E−05 | 1.0E+01 | 9.8E+00 | 3.4E−01 | 1.3E+00 |
| WRAP73 | 4.7E−04 | 9.3E+00 | 9.0E+00 | 3.4E−01 | 1.3E+00 |
| DOCK2 | 2.4E−04 | 1.3E+01 | 1.3E+01 | 3.4E−01 | 1.3E+00 |
| ALDOA | 2.0E−04 | 1.5E+01 | 1.4E+01 | 3.4E−01 | 1.3E+00 |
| FHOD1 | 4.0E−04 | 1.0E+01 | 1.0E+01 | 3.4E−01 | 1.3E+00 |
| SLC25A11 | 6.3E−05 | 1.0E+01 | 1.0E+01 | 3.4E−01 | 1.3E+00 |
| HDAC1 | 3.3E−04 | 1.1E+01 | 1.1E+01 | 3.4E−01 | 1.3E+00 |
| ALDH9A1 | 1.6E−04 | 1.1E+01 | 1.0E+01 | 3.3E−01 | 1.3E+00 |
| AP1M1 | 1.7E−04 | 1.1E+01 | 1.1E+01 | 3.3E−01 | 1.3E+00 |
| ACOX3 | 2.2E−04 | 7.8E+00 | 7.4E+00 | 3.3E−01 | 1.3E+00 |
| LSM14B | 2.6E−04 | 1.0E+01 | 9.9E+00 | 3.3E−01 | 1.3E+00 |
| UBE4B | 4.0E−04 | 1.1E+01 | 1.1E+01 | 3.3E−01 | 1.3E+00 |
| DDX42 | 4.2E−04 | 1.1E+01 | 1.1E+01 | 3.3E−01 | 1.3E+00 |
| PSMA7 | 2.1E−04 | 1.1E+01 | 1.1E+01 | 3.2E−01 | 1.3E+00 |
| POLR2G | 8.1E−05 | 1.0E+01 | 9.7E+00 | 3.2E−01 | 1.3E+00 |
| TCEANC2 | 1.4E−04 | 8.1E+00 | 7.8E+00 | 3.2E−01 | 1.3E+00 |
| MICU1 | 6.3E−05 | 1.1E+01 | 1.1E+01 | 3.2E−01 | 1.2E+00 |
| F8A1 | 1.2E−04 | 9.9E+00 | 9.6E+00 | 3.2E−01 | 1.2E+00 |
| AHCYL1 | 2.0E−04 | 1.1E+01 | 1.1E+01 | 3.2E−01 | 1.2E+00 |
| C16orf70 | 1.8E−04 | 9.4E+00 | 9.1E+00 | 3.2E−01 | 1.2E+00 |
| TMCO6 | 2.3E−04 | 8.6E+00 | 8.2E+00 | 3.2E−01 | 1.2E+00 |
| EIF4H | 9.2E−05 | 1.3E+01 | 1.2E+01 | 3.2E−01 | 1.2E+00 |
| PSMD13 | 3.0E−04 | 1.1E+01 | 1.1E+01 | 3.2E−01 | 1.2E+00 |
| ATP5J | 5.0E−04 | 9.6E+00 | 9.3E+00 | 3.1E−01 | 1.2E+00 |
| STK11IP | 2.3E−04 | 9.4E+00 | 9.0E+00 | 3.1E−01 | 1.2E+00 |
| SLC9A1 | 1.6E−04 | 1.1E+01 | 1.1E+01 | 3.1E−01 | 1.2E+00 |
| SAP18 | 1.3E−04 | 1.1E+01 | 1.1E+01 | 3.0E−01 | 1.2E+00 |
| FAM32A | 4.3E−04 | 1.1E+01 | 1.1E+01 | 3.0E−01 | 1.2E+00 |
| CORO1C | 3.8E−04 | 1.2E+01 | 1.2E+01 | 3.0E−01 | 1.2E+00 |
| KIF1C | 2.2E−04 | 1.0E+01 | 9.9E+00 | 3.0E−01 | 1.2E+00 |
| SPECC1L | 3.3E−04 | 1.0E+01 | 9.8E+00 | 3.0E−01 | 1.2E+00 |
| RNF20 | 4.2E−04 | 1.1E+01 | 1.1E+01 | 2.9E−01 | 1.2E+00 |
| BCORL1 | 4.3E−04 | 9.9E+00 | 9.6E+00 | 2.9E−01 | 1.2E+00 |
| MAP3K7 | 4.7E−04 | 9.8E+00 | 9.5E+00 | 2.8E−01 | 1.2E+00 |
| SURF4 | 8.1E−05 | 1.2E+01 | 1.1E+01 | 2.8E−01 | 1.2E+00 |
| VPS26B | 1.2E−04 | 1.1E+01 | 1.1E+01 | 2.8E−01 | 1.2E+00 |
| FKBP1A | 3.9E−04 | 1.3E+01 | 1.3E+01 | 2.8E−01 | 1.2E+00 |
| RAE1 | 3.0E−04 | 9.6E+00 | 9.4E+00 | 2.8E−01 | 1.2E+00 |
| RIC8A | 5.0E−04 | 1.1E+01 | 1.1E+01 | 2.7E−01 | 1.2E+00 |
| CERS2 | 4.7E−04 | 1.2E+01 | 1.2E+01 | 2.7E−01 | 1.2E+00 |
| STX5 | 1.2E−04 | 1.0E+01 | 1.0E+01 | 2.7E−01 | 1.2E+00 |
| PRKACA | 9.8E−05 | 1.2E+01 | 1.2E+01 | 2.7E−01 | 1.2E+00 |
| FDFT1 | 4.7E−04 | 1.1E+01 | 1.1E+01 | 2.7E−01 | 1.2E+00 |
| TRAPPC3 | 4.7E−04 | 1.0E+01 | 9.7E+00 | 2.7E−01 | 1.2E+00 |
| ALAS1 | 3.4E−04 | 1.0E+01 | 1.0E+01 | 2.6E−01 | 1.2E+00 |
| FAF2 | 2.2E−04 | 1.0E+01 | 9.9E+00 | 2.6E−01 | 1.2E+00 |
| EIF4ENIF1 | 1.9E−04 | 9.3E+00 | 9.0E+00 | 2.6E−01 | 1.2E+00 |
| AMMECR1L | 4.9E−04 | 9.1E+00 | 8.9E+00 | 2.6E−01 | 1.2E+00 |
| BCAS3 | 9.8E−05 | 9.2E+00 | 8.9E+00 | 2.5E−01 | 1.2E+00 |
| FBXO42 | 1.2E−04 | 9.5E+00 | 9.3E+00 | 2.5E−01 | 1.2E+00 |
| RNF216 | 4.0E−04 | 1.1E+01 | 1.0E+01 | 2.4E−01 | 1.2E+00 |
| COPS7A | 2.0E−04 | 9.8E+00 | 9.5E+00 | 2.4E−01 | 1.2E+00 |
| FXR2 | 3.3E−04 | 9.8E+00 | 9.6E+00 | 2.4E−01 | 1.2E+00 |
| CAPZB | 1.3E−04 | 1.3E+01 | 1.3E+01 | 2.4E−01 | 1.2E+00 |
| SH3KBP1 | 2.0E−04 | 1.3E+01 | 1.2E+01 | 2.3E−01 | 1.2E+00 |
| HNRNPL | 1.5E−04 | 1.2E+01 | 1.2E+01 | 2.3E−01 | 1.2E+00 |
| ARHGAP1 | 6.9E−05 | 1.2E+01 | 1.2E+01 | 2.3E−01 | 1.2E+00 |
| VPS4A | 3.0E−04 | 1.1E+01 | 1.0E+01 | 2.3E−01 | 1.2E+00 |
| TRAF7 | 4.3E−04 | 1.0E+01 | 1.0E+01 | 2.3E−01 | 1.2E+00 |
| DSCR3 | 3.8E−04 | 1.1E+01 | 1.1E+01 | 2.2E−01 | 1.2E+00 |
| ATP6VOA1 | 4.0E−04 | 1.1E+01 | 1.0E+01 | 2.2E−01 | 1.2E+00 |
| GYS1 | 3.4E−04 | 9.9E+00 | 9.6E+00 | 2.2E−01 | 1.2E+00 |
| PFN1 | 1.1E−04 | 1.5E+01 | 1.4E+01 | 2.2E−01 | 1.2E+00 |
| PIGS | 2.3E−04 | 1.1E+01 | 1.0E+01 | 2.2E−01 | 1.2E+00 |
| SLC35E1 | 3.4E−04 | 1.0E+01 | 1.0E+01 | 2.1E−01 | 1.2E+00 |
| CTCF | 1.3E−04 | 1.1E+01 | 1.1E+01 | 1.9E−01 | 1.1E+00 |
| KAT7 | 2.6E−04 | 1.0E+01 | 1.0E+01 | 1.9E−01 | 1.1E+00 |
| PPP1CA | 1.5E−04 | 1.2E+01 | 1.2E+01 | 1.9E−01 | 1.1E+00 |
| FNBP1 | 1.2E−04 | 1.3E+01 | 1.2E+01 | 1.9E−01 | 1.1E+00 |
| PRKCSH | 3.3E−04 | 1.2E+01 | 1.2E+01 | 1.6E−01 | 1.1E+00 |
| NRBP1 | 3.0E−04 | 1.2E+01 | 1.1E+01 | 1.4E−01 | 1.1E+00 |
| PSKH1 | 1.5E−04 | 9.2E+00 | 9.1E+00 | 1.3E−01 | 1.1E+00 |
| CSNK2B | 3.6E−04 | 1.1E+01 | 1.1E+01 | 1.2E−01 | 1.1E+00 |
| CS | 1.2E−04 | 1.1E+01 | 1.1E+01 | 1.1E−01 | 1.1E+00 |
| HDAC3 | 2.9E−04 | 1.0E+01 | 1.0E+01 | 1.1E−01 | 1.1E+00 |
| USP21 | 2.9E−04 | 8.9E+00 | 8.8E+00 | 8.0E−02 | 1.1E+00 |
| DCTN1 | 2.3E−04 | 1.1E+01 | 1.1E+01 | 5.8E−02 | 1.0E+00 |
| SRPR | 2.2E−04 | 1.2E+01 | 1.2E+01 | −3.9E−05 | 1.0E+00 |
| CLPTM1 | 3.0E−04 | 1.1E+01 | 1.1E+01 | −9.5E−03 | 9.9E−01 |
| PNPT1 | 3.3E−04 | 8.0E+00 | 9.0E+00 | −9.2E−01 | 5.3E−01 |
| TTC21A | 3.6E−04 | 5.9E+00 | 6.9E+00 | −1.0E+00 | 5.0E−01 |
| HERC6 | 4.4E−04 | 8.7E+00 | 1.0E+01 | −1.3E+00 | 4.0E−01 |
| AGRN | 2.0E−04 | 6.7E+00 | 8.4E+00 | −1.7E+00 | 3.1E−01 |
| IFI44 | 4.7E−04 | 1.0E+01 | 1.2E+01 | −1.8E+00 | 2.9E−01 |
| MX1 | 2.4E−04 | 1.2E+01 | 1.4E+01 | −1.9E+00 | 2.6E−01 |
| LY6E | 2.8E−04 | 1.2E+01 | 1.4E+01 | −2.0E+00 | 2.6E−01 |
| USP18 | 3.0E−04 | 6.5E+00 | 8.8E+00 | −2.3E+00 | 2.0E−01 |
| IFIT1 | 2.2E−04 | 1.1E+01 | 1.3E+01 | −2.7E+00 | 1.5E−01 |
| IFI44L | 3.0E−04 | 9.3E+00 | 1.2E+01 | −2.8E+00 | 1.4E−01 |
| SIGLEC1 | 1.3E−04 | 8.7E+00 | 1.2E+01 | −2.9E+00 | 1.3E−01 |
| IFI27 | 2.2E−04 | 8.3E+00 | 1.1E+01 | −3.1E+00 | 1.1E−01 |

These data strongly suggest that host peripheral blood gene expression patterns can be informative in the diagnosis of bacterial infection in LRTI.

### Example 2

### Transcriptomic Biomarkers to Discriminate Bacterial from Nonbacterial Infection in Adults Hospitalized with Respiratory Illness

The goal of the present study was to prospectively validate using RNA sequencing the classifier genes, as well as explore potentially new gene expression patterns to discriminate bacterial from viral LRTI in hospitalized adults.

The materials and methods employed in these experiments are now described.

Patient Population

Adults 21 years or older admitted to Rochester General Hospital (RGH), Rochester, New York, with diagnoses or symptoms compatible with acute LRTI from January through June 2013 using the same criteria as Suarez (Suarez NM, et al., 2015, J Infect Dis., 212:213-222). Admission logs were screened daily for patients with diagnoses of acute exacerbation of chronic obstructive pulmonary disease (AECOPD), bronchitis, asthma, influenza, viral syndrome, respiratory failure and congestive heart failure with infection, pneumonia or symptoms of wheezing, dyspnea, cough, sputum production, nasal congestion, sore throat, hoarseness. Patients were enrolled within 24 hours of admission and demographic, clinical and laboratory information collected. Exclusion criteria included antibiotic treatment before admission, immunosuppression, cavitary lung disease, and witnessed aspiration. The University of Rochester and RGH institutional review boards approved the study and written informed consent was obtained from subjects or authorized representatives. All study procedures were performed in accordance with the institutional policies and guidelines and regulations pertaining to research involving human subjects.

Microbiologic Methods

Nose and throat swabs (NTS), sputum, urine, and blood samples obtained at admission for bacterial and viral detection were processed at RGH clinical laboratories, as described (Falsey et al., 2013, J Infect Dis 207:432-41; Suarez et al., 2015, J Infect Dis 212:213-22). Briefly, single blood cultures positive for organisms consistent with skin flora (coagulase negative staphylococcus, Corynebacterium, alpha hemolytic streptococci, Propionibacterium acnes) were considered contaminants. Sputum cultures were considered positive if ≧2+ of a pathogenic bacterium grew from an adequate sample using standard criteria. Urine was assayed for Streptococcus pneumoniae antigen using Binax NOW (Binax, Inc, Scarborough, Me.). NTS and sputum were tested using the real time multiplex PCR (FilmArray Respiratory Panel, Idaho Technologies, Inc, Salt Lake City, Utah) for detection of 15 viruses and 3 atypical bacteria. Subjects were only included in the analysis who had a microbiologic diagnosis and had adequate diagnostic testing. Illness definitions are listed in Table 2.

TABLE 2
Illness definitions

| | |
|---|---|
| Microbiologic | |
| Classification | |
| Virus | NTS or sputum sample positive for any virus by one |
| infection | of the following: RT-PCR [all viruses], and all |
| alone | tests for bacteria were negative. |
| Bacterial | Negative viral diagnostic tests and any of the following: |
| infection | (1) positive blood culture meeting criteria for a pathogen, |
| alone | (2) positive culture for a respiratory pathogen from an |
| | adequate sputum sample, (3) a positive urinary antigen |
| | test for Streptococcus pneumoniae or Legionella |
| | pneumophila, or (4) positive PCR for Mycoplasma |
| | pneumoniae, Chlamydophila pneumoniae or |
| | Bordetella pertussis. |
| Viral-Bacterial | Meets definition for bacterial infection and viral |
| Infection | infection |
| Adequate | Subjects with fever were required to have blood cultures |
| Microbiologic | prior to antibiotics and those with productive cough were |
| Assessment | required to have an adequate sputum sample obtained |
| | within 24 hours of admission and ≦6 hours after |
| | administration of antibiotics. If these criteria were not |
| | met subjects could not be considered bacterial negative. |
| Analysis | |
| Groups | |
| Bacterial | Subjects with bacterial infection alone and those with |
| | mixed bacterial - viral infection |
| Non-bacterial | Viral infection alone |

Molecular Methods

Approximately 12 ml of whole blood was collected in Tempus™ Blood RNA Tube at enrollment. Following centrifugation, RNA was isolated from the pellet using the Tempus Spin RNA Isolation Kit. For 10 subjects blood was collected in CPT tubes and RNA isolated from spin-purified PBMCs using the RNeasy mini kit. Total RNA was processed for globin reduction using GLOBINclear Human Kit.

For quantitative PCR, cDNA was synthesized from 250 ng RNA using iScript cDNA synthesis kit and quantitative PCR performed as described (Tsalik et al., 2016, Sci Transl Med 8:322ra311) using noncommercial assays, Table 3. Difference in gene expression was tested by Wilcoxon Rank test (p<0.05).

For RNAseq, cDNA libraries were generated using 200 ng of globin-reduced total RNA from each sample. Library construction was performed using the TruSeq Stranded mRNA library kit (Illumina, San Diego, Calif.). cDNA quantity was determined with the Qubit Flourometer (Life Technologies, Grand Island, N.Y.) and quality assessed using the Agilent Bioanalyzer 2100 (Santa Clara, Calif.). Libraries were sequenced (single end reads) on the Illumina HiSeq2500 (Illumina, San Diego, Calif.) to generate 20 million reads/sample.

Reads were aligned using the TopHat algorithm and expression values summarized using HTSeq (Kim et al., 2013, Genome Biol 14:R36). Raw counts were normalized using Conditional Median normalization. Differences in expression between bacterial and non-bacterial infected subjects for each gene were assessed by Wilcoxon rank test at an FDR q<0.05.

Statistical Methods

LASSO-penalized logistic regression was used to select pathway-based genetic predictors of bacterial infection (see below). Model parameters were selected via cross-validation (CV), and the model's predictive ability was assessed by a nested cross-validated (NCV) estimate of the area under the ROC curve (AUC). Briefly, genes were univariately screened, and those with a nominal Wilcoxon p<0.10 were assigned to 1330 known canonical pathways, as defined in the Molecular Signature Database (MSigDB) of Broad Institute (Anders et al., 2015, Bioinformatics 31:166-9). The first principal component (PC1) of the genes in each pathway was derived, and genes with loadings close to 0 were removed. Pathways were subjected to an additional univariate Wilcoxon screen with significance level selected by CV. LASSO was then applied to these pathway PC1s to obtain a logistic model with pathway predictors.

Clinical variables were dichotomized for robustness and ease of interpretation, univariately screened using nominal a=0.05, and all-subsets selection was used to build a clinical-only logistic regression model with the number of variables selected via CV.

Steps for Pathway Constrained PCA LASSO Predictor is described:

1. Screen genes univariately, dropping genes with nominal Wilcoxon p-value greater than 0.10.

2. Standardize genes to have mean 0 and SD 1 prior to forming pathway principal components (PC).

3. Create pathway principal components (PC) using the standardized screened genes.

4. Set to zero (hard-threshold) any loadings whose |loading|<0.75*mean(|loadings|) within that pathway.

5. Screen the pathway first hard-thresholded PC (HTPC) using a Bonferroni Wilcoxon p-value (with alpha selected by CV).

6. Standardize each pathway first HTPC to have mean 0 and SD 1 before using LASSO to estimate pathway OR.

7. Perform LASSO on the screened pathway first HTPC, selecting the LASSO penalty via CV (along with the screening level).

8. If any LASSO OR <1, take the reciprocal (so OR >=1) and swap the signs of all of the loadings for that pathway.

9. Shrink to one (hard-threshold) any LASSO OR <mean(OR)^{0.75}

Primer sequences

Primer sequences are shown in Table 3. Shown are gene ID, forward and reverse primer sequences or assay ID for commercial assays.

TABLE 3

| Gene | Forward Primer | Reverse Primer |
|---|---|---|
| SIGLEC10 | AGATTCTACCGAAGAGACGGAC (SEQ ID NO: 1) | CGTCGGGACCACATTGATGTA (SEQ ID NO: 2) |
| LTA4H | ATGAGTGCTATTCGTGATGGAGA (SEQ ID NO: 3) | TGGGCCAATTTGCCTGCTT (SEQ ID NO: 4) |
| AATF | CCAGGGTGATTGACAGGTTTG (SEQ ID NO: 5) | CCAGTTTTCTAATGCTACCCACT (SEQ ID NO: 6) |
| TNFAIP8L3 | GATTCTGAGCAAAATAGCCAGCA (SEQ ID NO: 7) | GGCTTCCTTCTTGTTGTGTGT (SEQ ID NO: 8) |
| SIGLEC1 | CCACTAGGGCTGATACTGGCT (SEQ ID NO: 9) | GAGGCGGGTGGTTGACTAC (SEQ ID NO: 10) |
| C11orf82 | GTTGTCCACCTTCGTTACTCAG (SEQ ID NO: 11) | GAGAATGGCAGATCATCCCAAAT (SEQ ID NO: 12) |
| FAM101B | AGTGGAGTTTGACCCCTTACC (SEQ ID NO: 13) | GAAGTGCCTCTCGGAGTCGTA (SEQ ID NO: 14) |
| PPM1N | CGAGCGTTGGGCGACTTTA (SEQ ID NO: 15) | CAGGAGCATGAACTCGTCCTC (SEQ ID NO: 16) |
| PCOLCE2 | TACTTGGAAAATCACAGTTCCCG (SEQ ID NO: 17) | CGGCACAGGTTGTCACTCTC (SEQ ID NO: 18) |
| AGRN | GTCCTGCGTCTGCAAGAAGAG (SEQ ID NO: 19) | CTCGCATTCGTTGCTGTAGG (SEQ ID NO: 20) |
| IFI27 | TGCTCTCACCTCATCAGCAGT (SEQ ID NO: 21) | CACAACTCCTCCAATCACAACT (SEQ ID NO: 22) |
| RSAD2 | TTGGACATTCTCGCTATCTCCT (SEQ ID NO: 23) | AGTGCTTTGATCTGTTCCGTC (SEQ ID NO: 24) |
| OA52 | CTCAGAAGCTGGGTTGGTTTAT (SEQ ID NO: 25) | TTTATCGAGGATGTCACGTTGG (SEQ ID NO: 26) |
| IFIT3 | AGAAAAGGTGACCTAGACAAAGC (SEQ ID NO: 27) | CCTTGTAGCAGCACCCAATCT (SEQ ID NO: 28) |
| IFI44 | GGTGGGCACTAATACAACTGG (SEQ ID NO: 29) | CACACAGAATAAACGGCAGGTA (SEQ ID NO: 30) |
| OASL | CTGATGCAGGAACTGTATAGCAC (SEQ ID NO: 31) | CACAGCGTCTAGCACCTCTT (SEQ ID NO: 32) |
| KAL1 | CCTGCAAGGAATCAGGGGAC (SEQ ID NO: 33) | GTCAAGCATTCGTAGCTCTTCT (SEQ ID NO: 34) |
| MX1 | AGCGGGATCGTGACCAGAT (SEQ ID NO: 35) | TGACCTTGCCTCTCCACTTATC (SEQ ID NO: 36) |
| GPR84 | TTGGCATCTTCTATTGCCTCATC (SEQ ID NO: 37) | TGTCGCAACTTGTATTGGTCC (SEQ ID NO: 38) |
| USP18 | AACGTGCCCTTGTTTGTCCAA (SEQ ID NO: 39) | GAGTCCTTCACCCGGATCGTA (SEQ ID NO: 40) |
| IFIT1 | GCGCTGGGTATGCGATCTC (SEQ ID NO: 41) | CAGCCTGCCTTAGGGGAAG (SEQ ID NO: 42) |
| IFI44L | AGCCGTCAGGGATGTACTATAAC (SEQ ID NO:43) | AGGGAATCATTTGGCTCTGTAGA (SEQ ID NO: 44) |

The results of the experiments are now described.

Demographics and Illness Characteristics

Two hundred and thirteen patients were enrolled; 100 had definitive microbiologic diagnoses and 94 generated transcriptomic data passing quality control metrics. Clinical characteristics of these 94 subjects are shown in Table 4. Mean age was 61 years, 55% were female and 78% were Caucasian. The majority (98%) had at least one chronic medical condition, 7% required intensive care and none died. Clinical diagnoses included asthma exacerbation (n=17), bronchitis (n=25), AECOPD (n=21), pneumonia (n=23) and bacteremia (n=8). For molecular analyses, 41 subjects were considered “bacterial” infection (27 bacterial only and 14 mixed viral/bacterial) and 53 subjects were classified “non-bacterial” infection (viral infection alone).

TABLE 4
Clinical Variables

| | | | |
|---|---|---|---|
| | Bacterial | | |
| | (Bacterial | | |
| | alone and | Non | |
| | Mixed | Bacterial | Fisher's |
| | Bacterial | (Viral | Exact or |
| All | Viral) | Alone) | t-test |
| N = 94 | N = 41 | N = 53 | (P value) |

Demographics

| | | | | |
|---|---|---|---|---|
| Age, mean ± SD | 61 ± 18 | 67 ± 18 | 57 ± 17 | 0.01 |
| Male Sex, No. (%) | 42 (45) | 21 (51) | 21 (40) | 0.30 |
| White Race | 73 (78) | 35 (85) | 38 (72) | 0.14 |

Underlying Conditions

| | | | | |
|---|---|---|---|---|
| COPD, No (%) | 30 (32) | 17 (41) | 13 (25) | 0.12 |
| CHF, No. (%) | 23 (24) | 10 (24) | 13 (25) | 1 |
| Diabetes, No. (%) | 27 (29) | 13 (32) | 14 (26) | 0.65 |

Symptoms

| | | | | |
|---|---|---|---|---|
| Nasal Congestion, | 50 (53) | 14 (34) | 36 (68) | 0.002 |
| No. (%) | | | | |
| Cough, No. (%) | 85 (90) | 34 (83) | 51 (96) | 0.04 |
| Sputum, No. (%) | 66 (70) | 29 (71) | 37 (70) | 1 |
| Dyspnea, No. (%) | 81 (86) | 34 (83) | 47 (89) | 0.55 |
| Rigors, No. (%) | 30 (32) | 12 (29) | 18 (34) | 0.66 |

Physical Findings

| | | | | |
|---|---|---|---|---|
| Wheezing, No. (%) | 66 (70) | 25 (61) | 41 (77) | 0.11 |
| Rales, No. (%) | 29 (31) | 15 (37) | 14 (26) | 0.37 |
| Temperature (° C.) | 37.8 ± 1.0 | 37.8 ± 0.9 | 37.8 ± 1.0 | 0.93 |
| Systolic Blood | 112 ± 21 | 111 ± 25 | 113 ± 18 | 0.73 |
| Pressure, mean ± SD | | | | |
| Oxygen Saturation, | 90.0 ± 6.0 | 88.4 ± 7.1 | 91.2 ± 4.7 | 0.03 |
| mean ± SD | | | | |

Laboratory Data

| | | | | |
|---|---|---|---|---|
| Infiltrate on Chest | 27 (29) | 21 (52) | 6 (11) | <0.0001 |
| Radiograph | | | | |
| White blood cell | 10.7 ± 5.6 | 13.7 ± 6.8 | 8.5 ± 2.9 | <0.0001 |
| count, mean ± SD | | | | |
| % bands in peripheral | 3.1 ± 5.6 | 5.2 ± 6.9 | 0.7 ± 1.9 | 0.002 |
| blood, mean ± SD | | | | |
| Blood urea nitrogen, | 19 ± 12 | 23 ± 15 | 16 ± 8 | 0.005 |
| mean ± SD | | | | |

A wide range of pathogens were detected (Table 5) with influenza A the most common virus and Streptococcus pneumoniae the most common bacteria documented.

TABLE 5
Pathogens Identified

| | Number of | | Number of |
|---|---|---|---|
| Pathogen | detections | Pathogen | detections |
| Viruses | 68 | Bacteria | 44 |
| Adenovirus | 1 | Streptococcus pneumoniae | 14 |
| Coronaviruses | 12* | Haemophilus pneumoniae† | 9 |
| Influenza A | 22 | Staphylococcus aureus | 6 |
| Influenza B | 4 | Moraxella catarrhalis | 3 |
| Human | 11* | Beta hemolytic streptococci | 5 |
| metapneumovirus | | | |
| Parainfluenza | 2 | Chlamydophila pneumoniae | 3 |
| viruses | | | |
| Rhinoviruses | 9 | Legionella pneumophila† | 2 |
| Respiratory | 7 | Coagulase negative | 1 |
| Syncytial virus | | staphylococcus‡ | |
| | | Streptococcus salivarius‡ | 1 |

*One subject had mixed HMPV and coronavirus
†One subject had mixed Legionella and Haemophilus influenzae
‡Multiple blood cultures positive, source unclear

Transcriptomic Data Quality

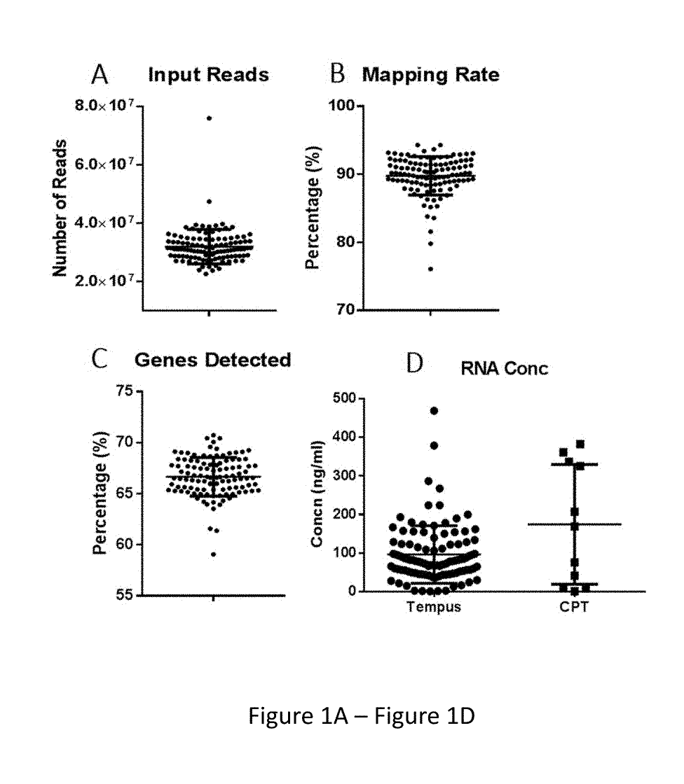

Six of the 100 samples were excluded based on poor RNA or sequence quality (read count/mapped read numbers) or if they were outliers in unsupervised cluster analysis, leaving 94 samples for analysis. On average 38±5.6 million reads were generated from each of the cDNA libraries using globin-reduced, unfractionated whole blood RNA, with a mapping rate of 89.8±2.8% indicating high quality sequence data. Genomic coverage averaged 66.1±6.5% of the human transcriptome (FIG. 1A, FIG. 1B) indicating appropriate diversity of transcript sampling. Of 25,559 mapped genes, 2,440 genes with zero counts across all subjects were excluded. In addition, 7,438 genes with normalized counts of <3 for >75% subjects were excluded leaving 15,681 genes for analysis (FIG. 1C). RNA concentration was similar for blood collected in CPT and Tempus tubes (FIG. 1D).

Replication of Array-based Predictors

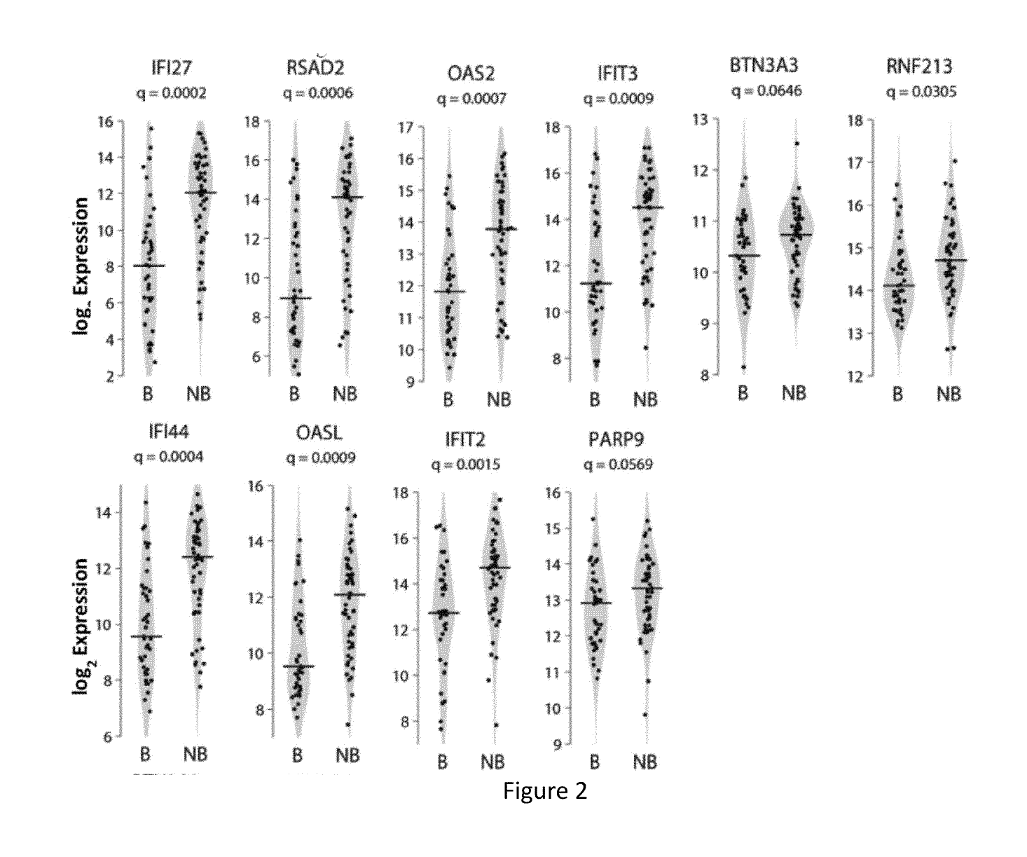

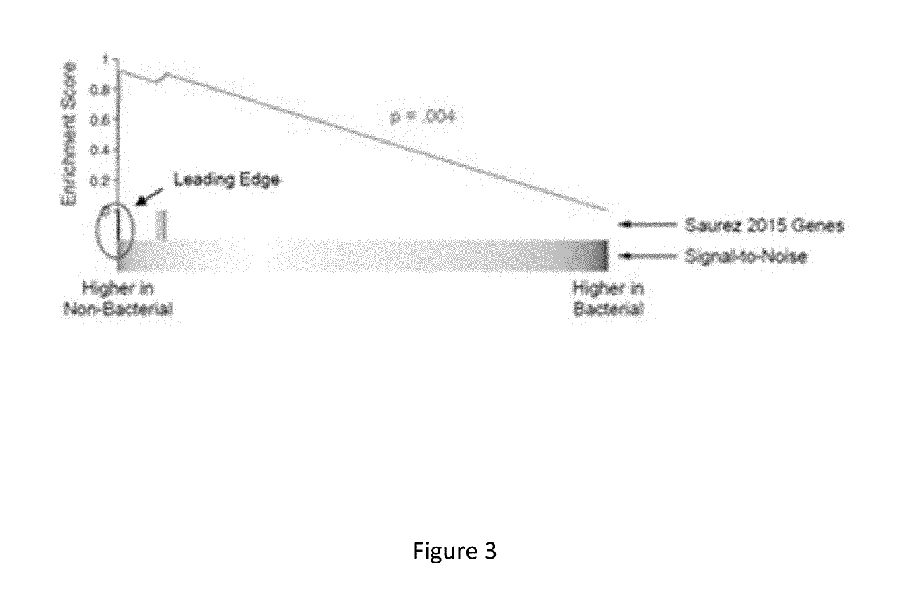

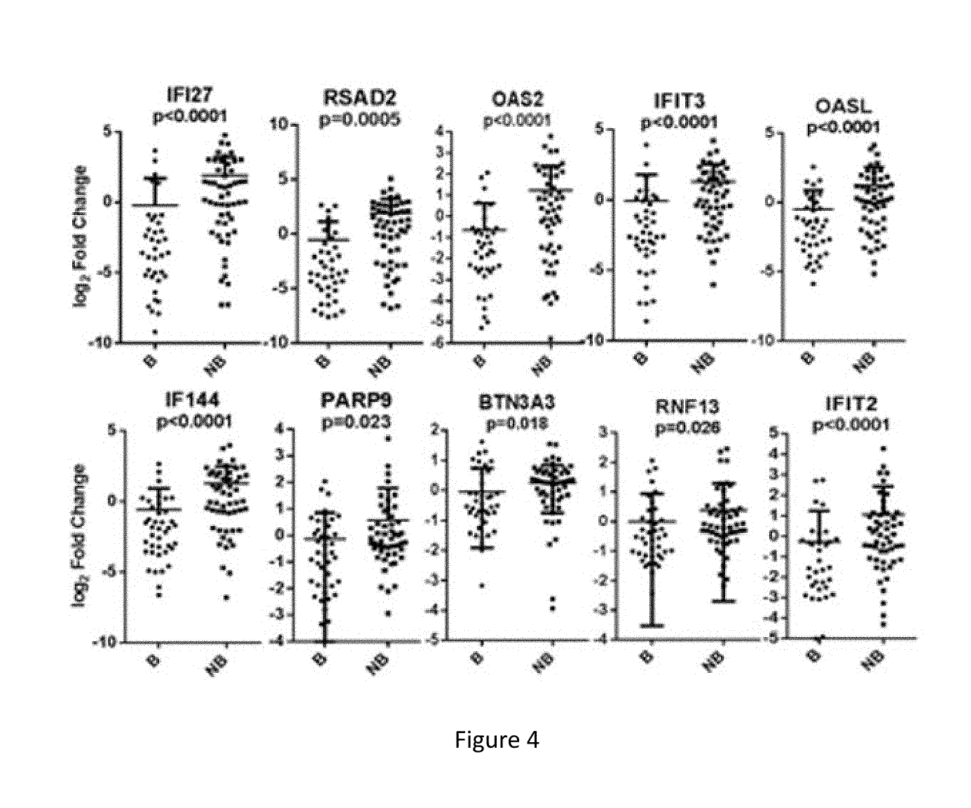

The expression of ten marker genes, previously identified by Suarez (Suarez et al., 2015, J Infect Dis 212:213-22), were assessed using RNA-Seq and qPCR for the ability to distinguish bacterial from non-bacterial illness. In the transcriptomic data, 8 of the 10 genes demonstrated significant differences between groups using Wilcoxon Rank test at a False Discovery Rate (FDR) or q<0.05 (FIG. 2). By qPCR all ten showed significant difference between bacterial and non-bacterial groups by Wilcoxon Rank test at a nominal p-value <0.05 (FIG. 4). Of note, increased expression of all 10 genes is associated with non-bacterial infection, with most belonging to the interferon family (FIG. 3). Gene Set Enrichment Analysis (GSEA) (Subramanian et al., 2004, PNAS 102:15545-50) using those ten genes as a gene set provided high enrichment scores (ES) from seven of those genes (IFI27, RSAD2, IFI44, IFIT3, OASL, OAS2, and IFIT2) in the leading edge of the plot indicating these genes are relatively informative for distinguishing groups.

Novel Markers for Bacterial Infection

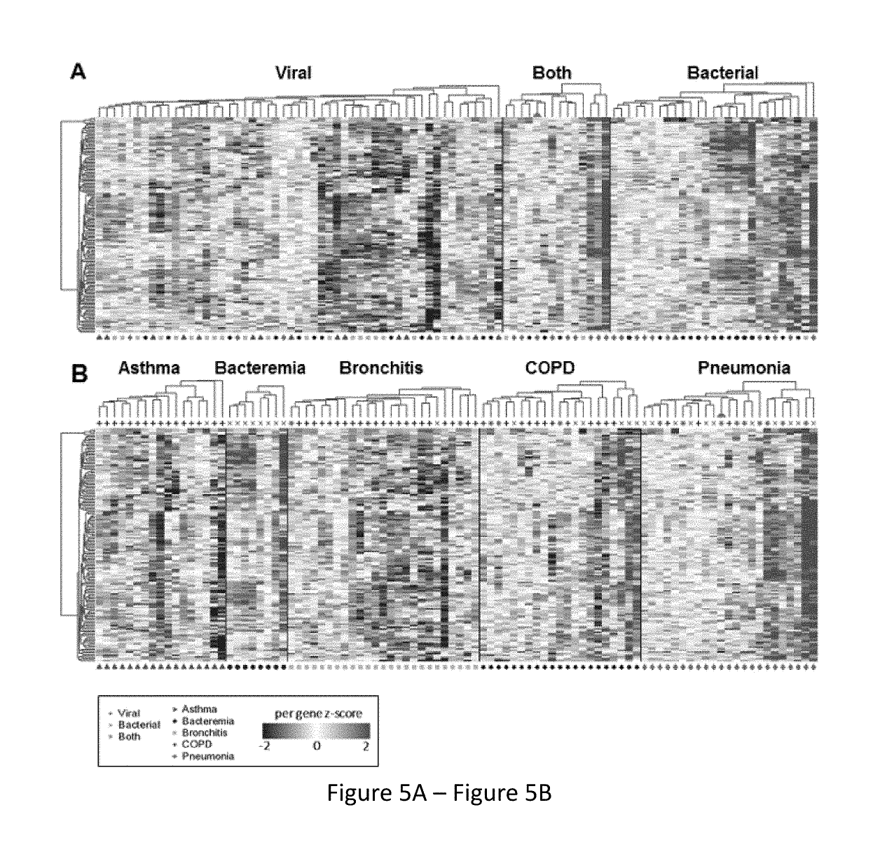

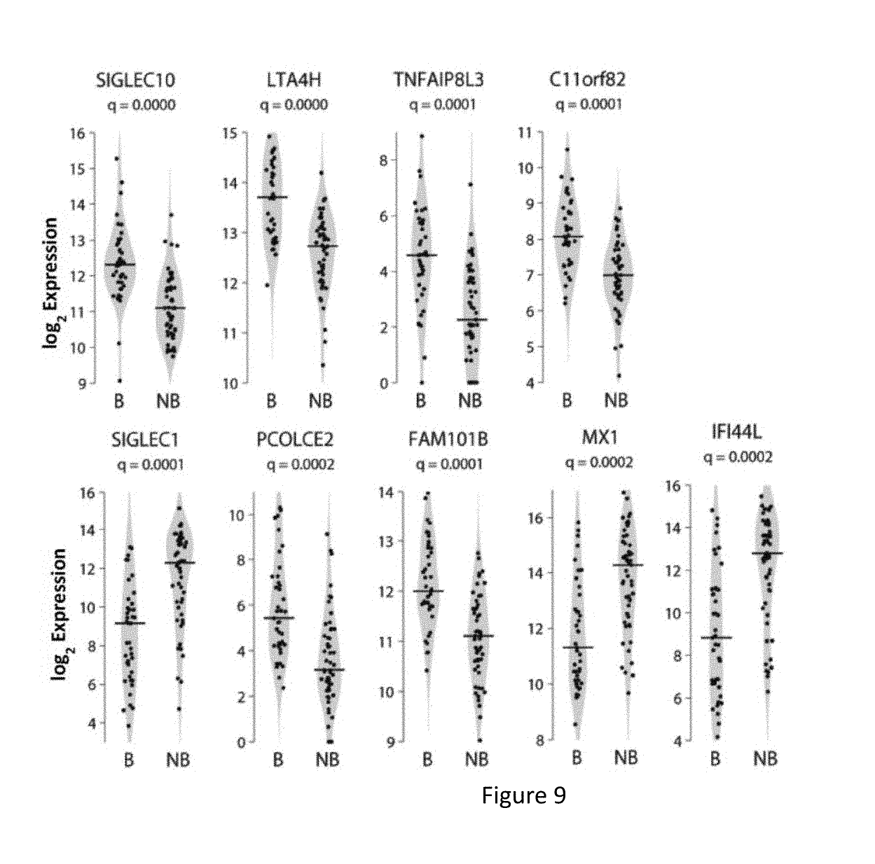

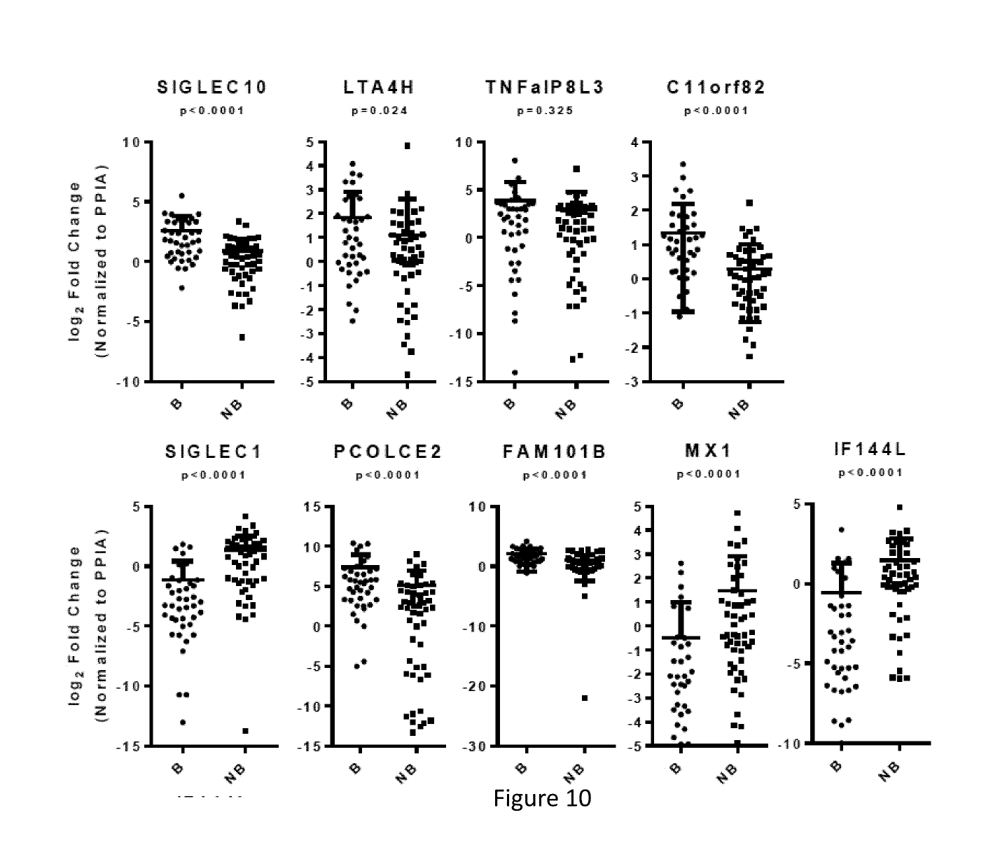

In addition to validating these previously identified markers for bacterial infection (Suarez et al., 2015, J Infect Dis 212:213-22), it was sought to identify novel gene sets with the greatest potential for accurate classification. Using the Wilcoxon rank test with FDR q<0.05, 141 genes were identified that are differentially expressed between subjects with bacterial versus non-bacterial infections (displayed in FIG. 5A and FIG. 5B). In contrast to the predictive markers replicated above, most of these 141 genes have higher expression in most subjects with bacterial infection. Notably, reduced expression of these 141 genes was observed in patients with a clinical diagnosis of asthma and bronchitis, whereas patients with pneumonia and bacteremia tended to have increased expression of these genes, with AECOPD presenting a mixed pattern. A subset of nine genes was selected for molecular validation by qPCR, based on their magnitude of difference or biological relevance (FIG. 9). Significant differences in expression were validated for 8 of the 9 genes (FIG. 10).

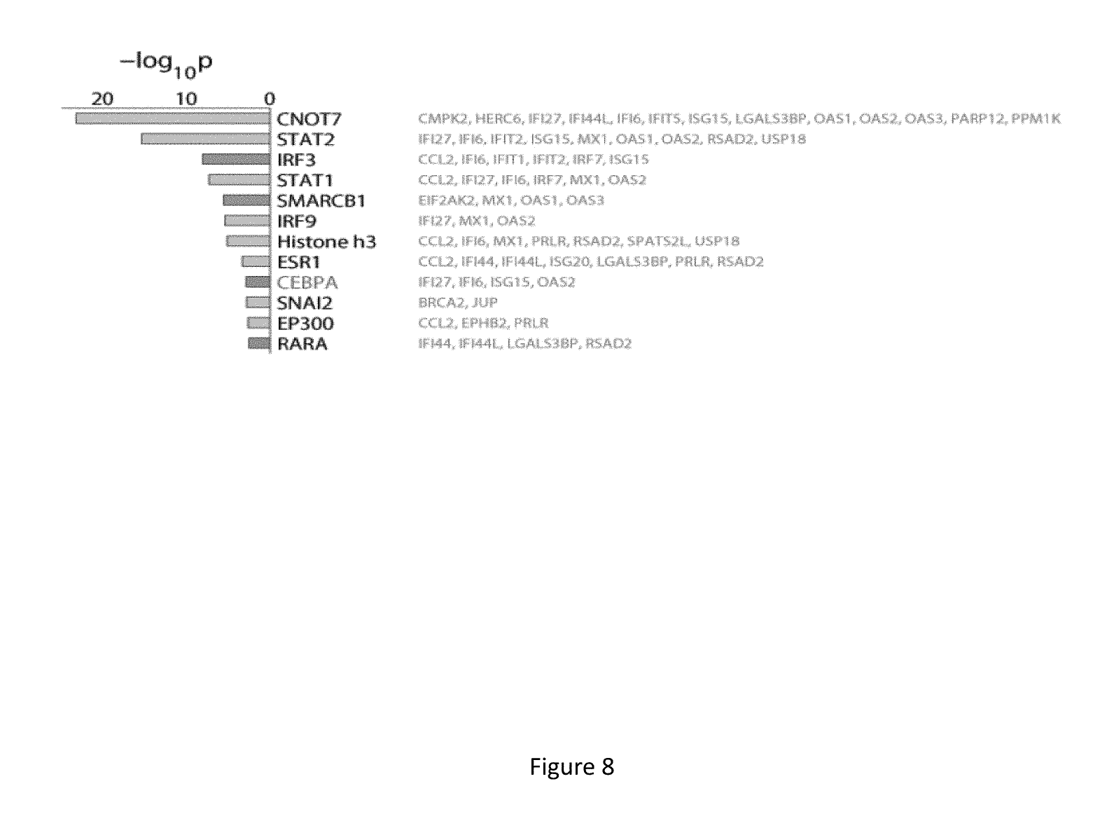

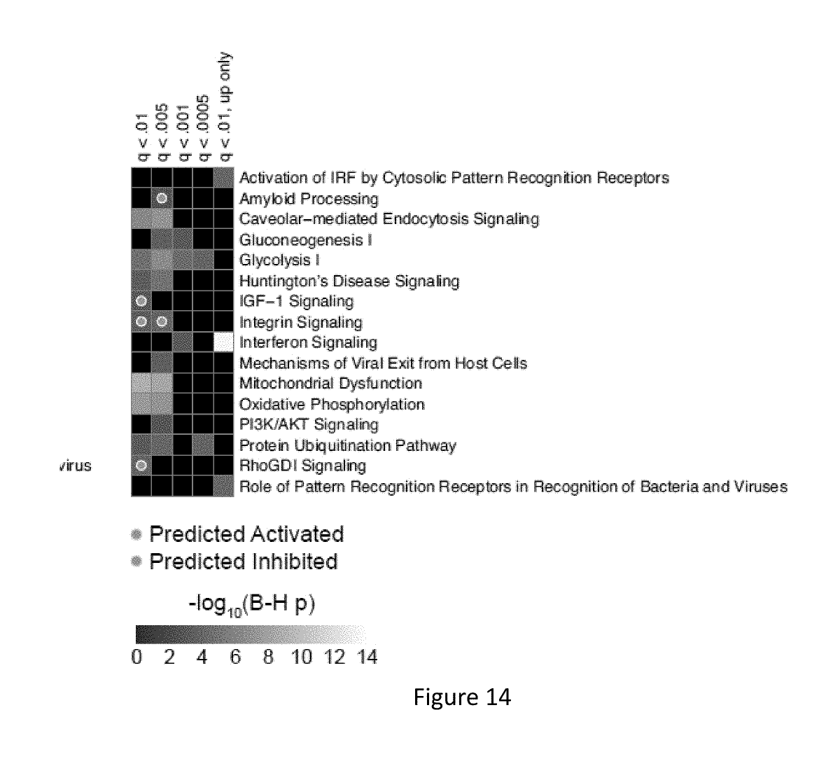

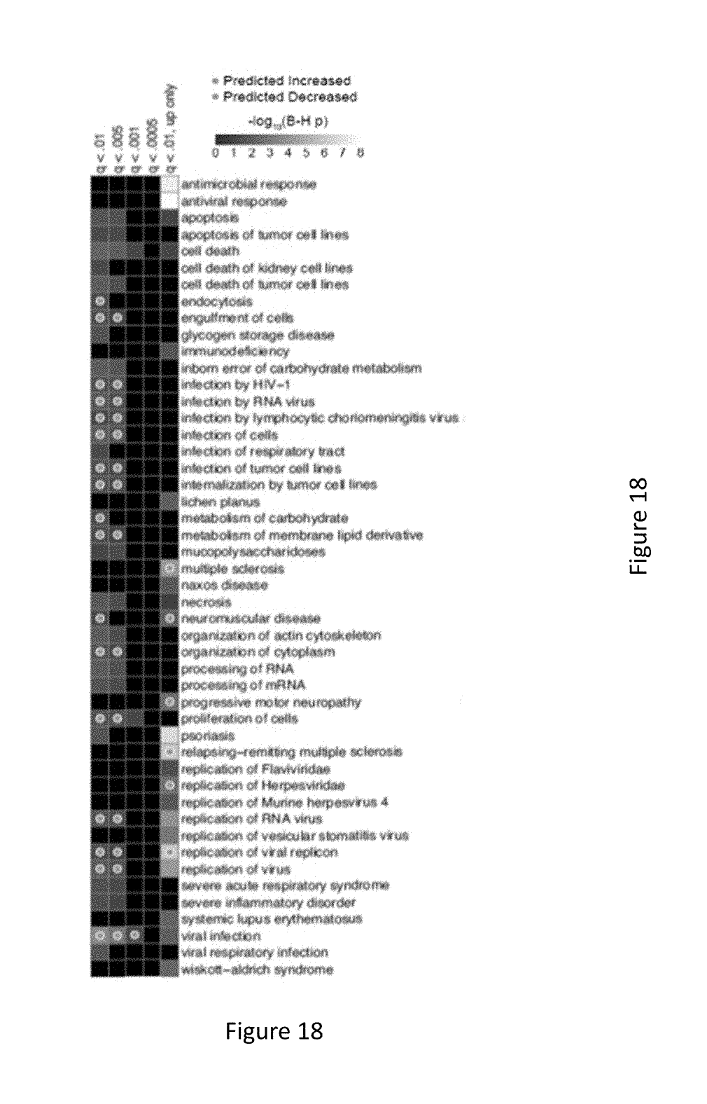

Ingenuity Pathway Analysis (IPA) was used to define the biology represented by genes differentially expressed between nonbacterial vs. bacterial infections (FIG. 18, FIG. 14, and FIG. 8). For this various differentially expressed gene sets were analyzed, based upon q-value thresholds; from q<0.01 (n=1434) to q<0.0005 (n=304). This analysis predicted inhibition of numerous viral infection and replication related biological functions (FIG. 18). Pathways analysis also implicated virus related pathways (interferon signaling, activation of IRF), as well as non-viral pathways (FIG. 14). Of note, inhibition of integrin signaling, activation of RhoGDI signaling and involvement (no direction predicted) of the IGF1 signaling pathways were also predicted. These analyses also sought to predict regulatory molecules that may drive differential expression distinguishing bacterial from non-bacterial infection subjects (FIG. 8). This implicated multiple regulators of interferon signaling (IRF3, IRF9, STAT1/2). The most significant regulator identified was CNOT7, reported to be responsible for dampening interferon signaling through STAT1 (Chapat et al., 2013, EMBO J 32:688-700).

Assessment of Gene Expression Markers for Bacterial LRTI

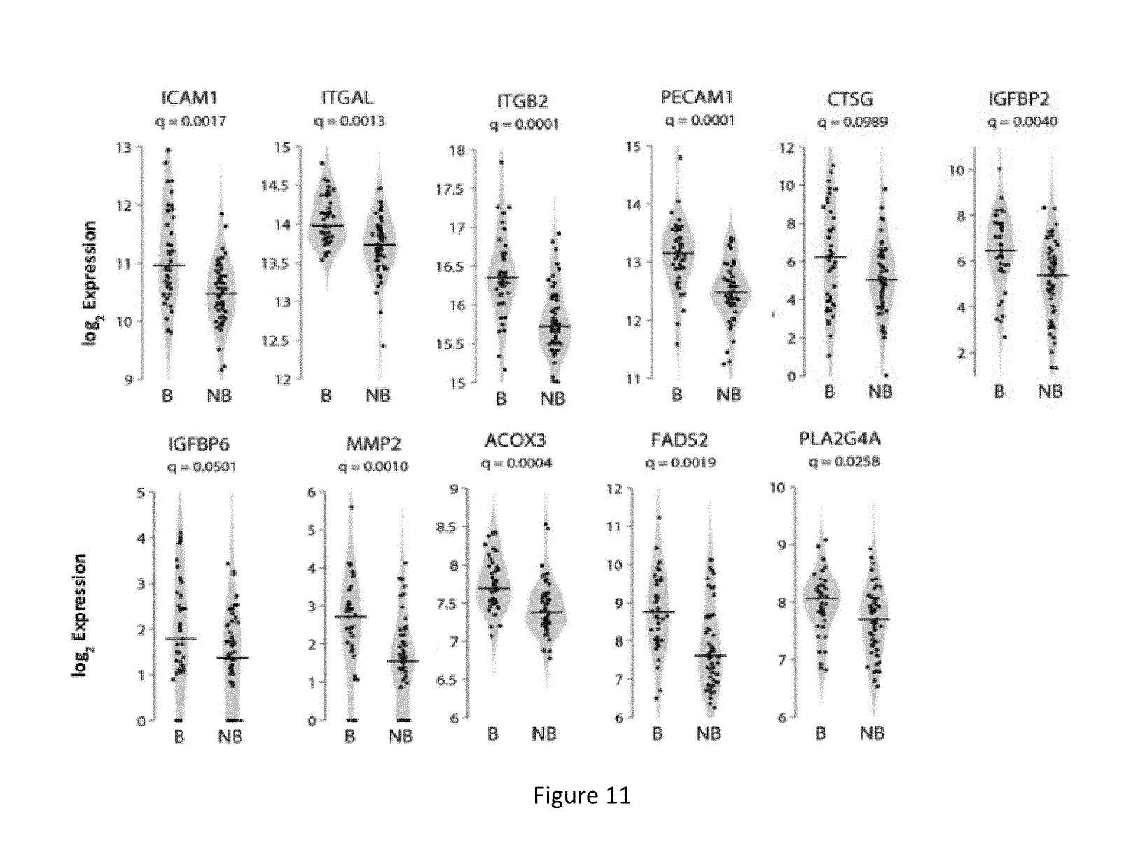

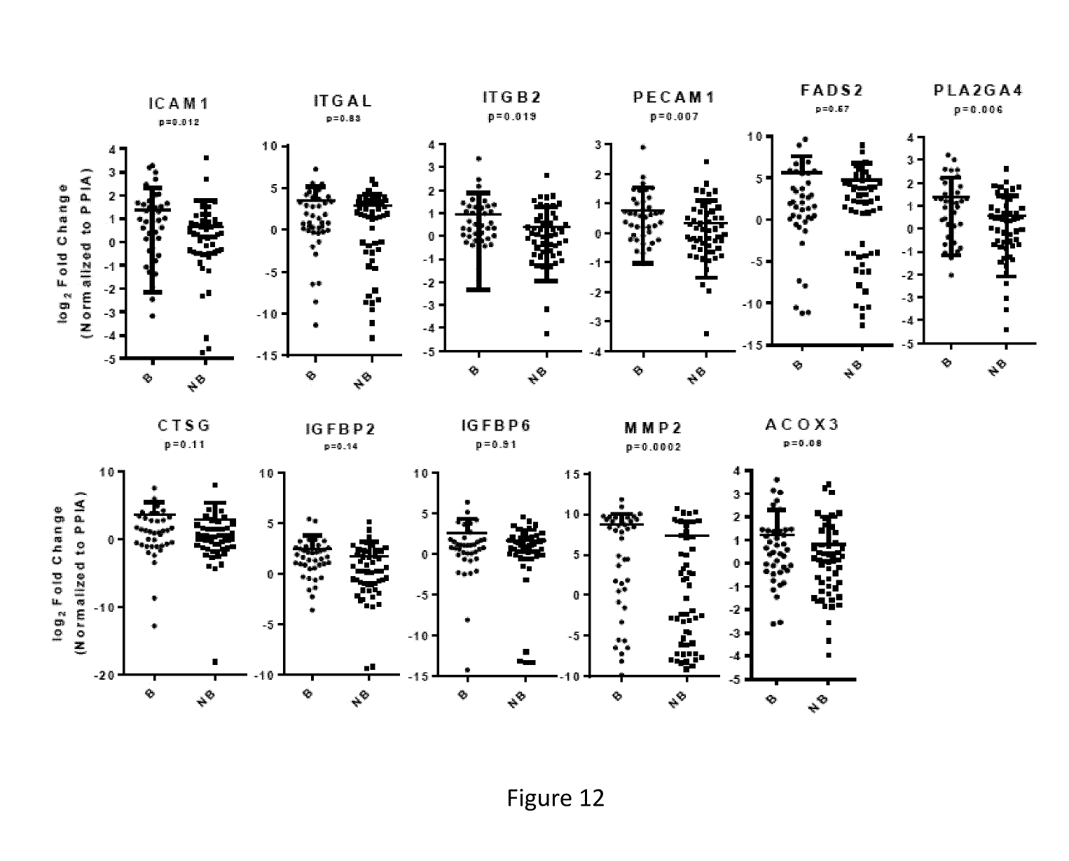

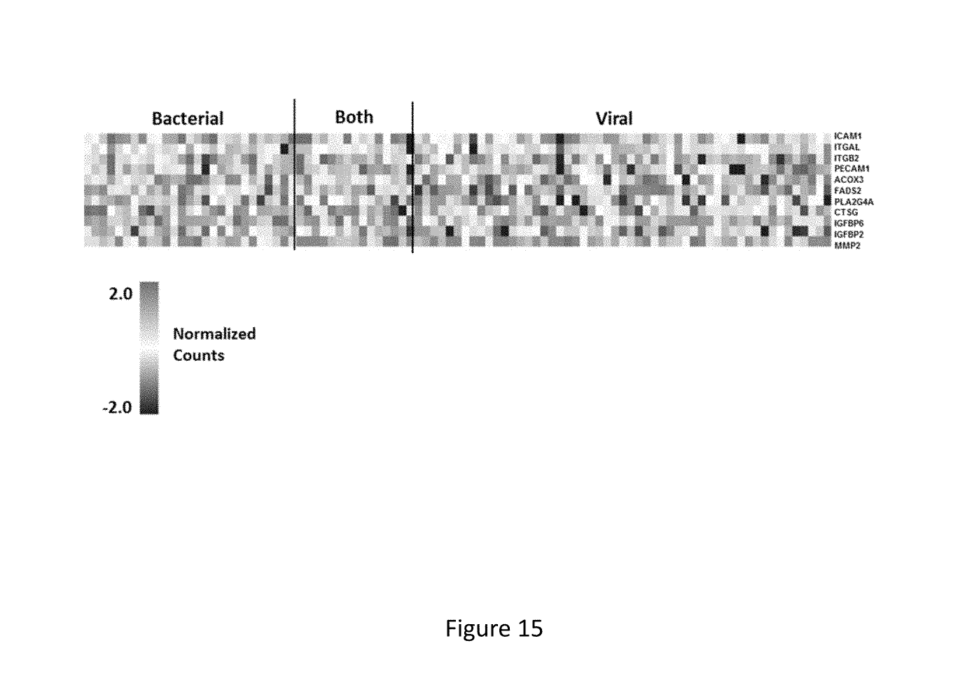

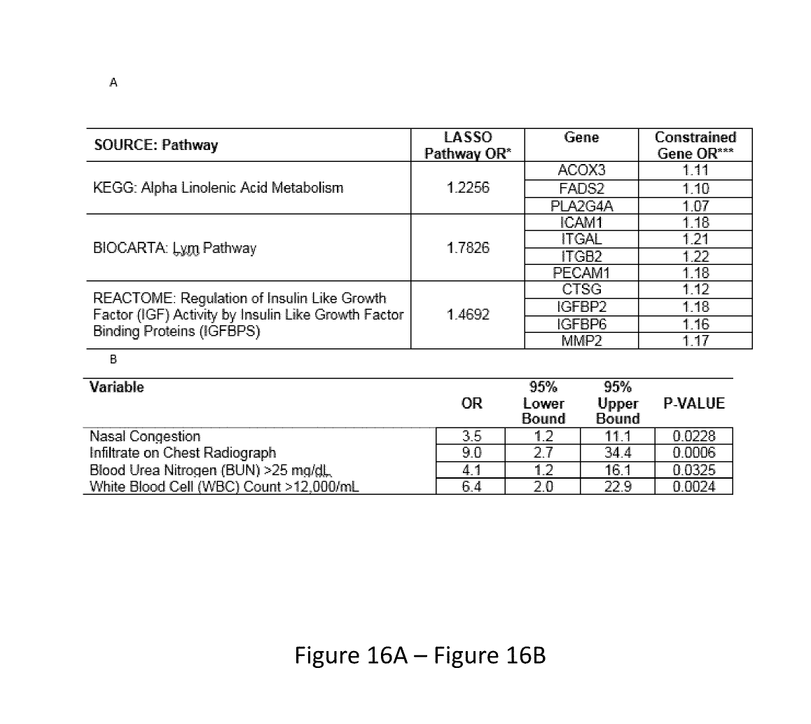

A set of genes whose expression may be useful for classification of bacterial versus non-bacterial infections were identified. Biological priors were leveraged as a means of identifying the most robust predictors since (1) expression changes at the individual gene level alone may not be sufficient to identify biologically meaningful data and (2) there exist substantial statistical advantages to dimension reduction strategies in the analysis of genome-wide data. A curated list of genes (Subramanian et al., 2004, PNAS 102:15545-50) was used to partition the transcriptomic data into 1330 biologically-relevant gene sets, or pathways. Gene expression data from the genes in each pathway were reduced to a single derived pathway variable as described in the Statistical Methods. Cross-validation simultaneously selected a LASSO penalty parameter and a Bonferroni-corrected significance level of 0.05 for screening pathways, at which 43 pathways were univariately associated with infection status. Of those, LASSO-penalized logistic regression retained 3 pathways consisting of a total of 11 genes (FIG. 11) providing the greatest predictive value for classifying subjects as bacterial or non-bacterial. Differential expression was confirmed for 6 genes by qPCR (FIG. 12). A Heat map demonstrating the differential expression of 11 selected genes between subjects with bacterial, mixed viral bacterial and viral infection alone is shown in FIG. 15. Pathway and gene names, pathway odds ratios (OR) based upon LASSO and constrained gene OR are presented in FIG. 16A. Sensitivity-specificity analysis indicated models using these gene sets provided a naive area under the receiver-operator curve (AUC) of 0.94, and a conservative, fully nested, cross-validated (NCV) AUC of 0.86.

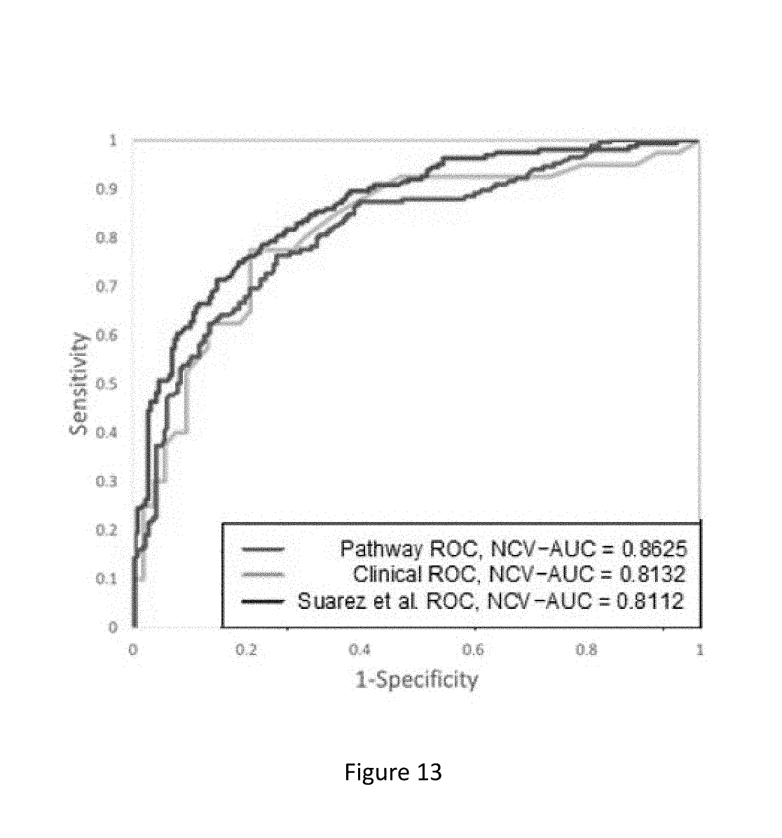

Clinical variables were also tested for the ability to distinguish subjects with bacterial versus non-bacterial infection using the screened all subsets selection method described in the Statistical Methods. Cross-validation chose a 4-predictor model consisting of nasal congestion, infiltrates on chest radiograph, blood urea nitrogen levels and white blood cell count (FIG. 16B). This clinical model had a surprisingly robust naäve AUC of 0.833 and an NCV-AUC of 0.813 (FIG. 13). Biomarker selection was also optimized by combining clinical and gene expression data. All such models failed to select any clinical variables.

Finally, a model was fit using only the 10 genes previously identified by Suarez (Suarez et al., 2015, J Infect Dis 212:213-22). Using LASSO-penalized logistic regression, a stable 5-gene model was identified with an NCV-AUC of 0.811. Therefore, this novel pathway-based biomarkers out-performed both the set of previously implicated genes and clinical variables (FIG. 13).

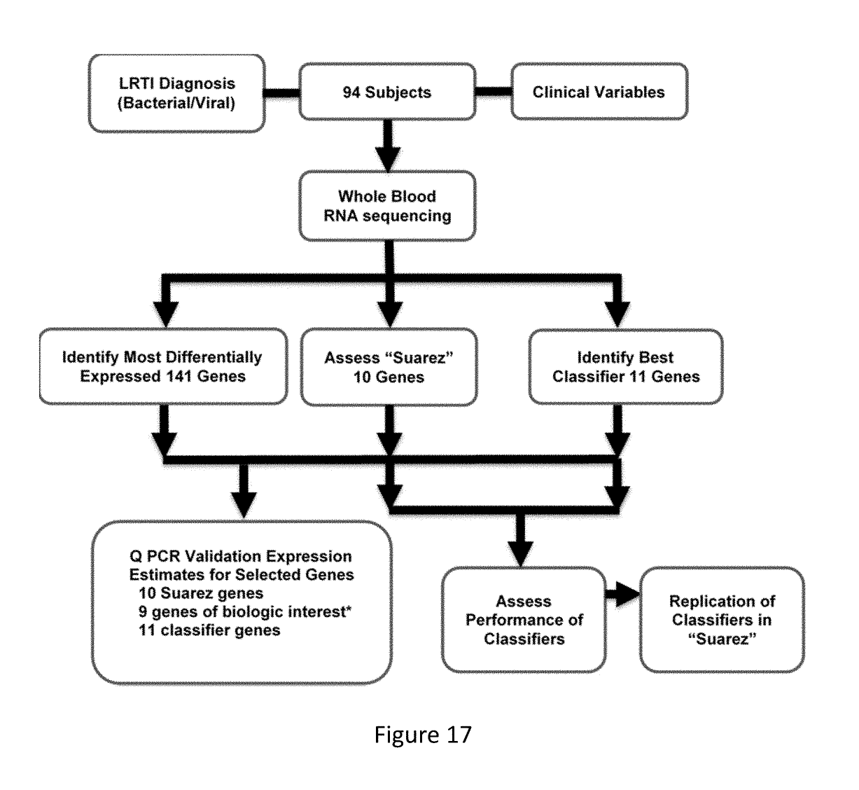

To summarize the different analyses, a flow chart of the analysis of the different gene sets derived from RNA sequencing data from the 94 study subjects is provided in FIG. 17. First, the expression of 10 genes identified by Suarez et. al. was assessed to be differentially expressed comparing bacterial to viral infections. Next, the 141 most differentially-expressed genes were identified, as defined by statistical differences in bacterial vs. non-bacterial infection. Separately, a pathway-based approach was used to develop a novel gene expression classifier for discriminating bacterial vs. non-bacterial infection. An additional 9 genes of biologic interest were selected from the 141gene set after considering the 10 Suarez genes. Validation of RNAseq-based expression estimates for selected genes was attempted by qPCR. Finally, performance of the novel 3 pathway-based 11 gene classifier was assessed in this cohort.

A Molecular Classifier for Bacterial LRTI

To classify subjects as bacterial or non-bacterial, it is necessary to threshold a molecular predictor. The sensitivity and specificity associated with several candidate thresholds was estimated for the nominal predicted probability of a bacterial infection (Table 6). A threshold with sensitivity >specificity >70% was targeted, corresponding with weighting errors among the bacterial subjects 50% higher than those among the non-bacterial subjects.. The optimal threshold was estimated to be about 0.40, with a naive sensitivity of 90% and specificity of 83%, and a NCV sensitivity of 79% and specificity of 76%.

TABLE 6
Thresholds for the nominal predicted probability of a bacterial infection

| | Naïve Results | | | | | |
|---|---|---|---|---|---|---|
| | | | # Bacterial | # Not | | |
| | | | Classified | Bacterial | Nested | |
| | | | as Not | Classified | Cross-Validated | |
| Cutoff | Sensitivity | Specificity | Bacterial | as Bacterial | Sensitivity | Specificity |
| 0.35 | 0.951 | 0.679 | 2 | 17 | 0.854 | 0.670 |
| 0.40 | 0.902 | 0.830 | 4 | 9 | 0.793 | 0.759 |
| 0.45 | 0.805 | 0.887 | 8 | 6 | 0.744 | 0.816 |
| 0.50 | 0.707 | 0.962 | 12 | 2 | 0.665 | 0.868 |

A particularly challenging group of subjects are those with mixed viral bacterial infections. Of the 14 subjects with mixed infection 12 (naive sensitivity, 85.7%) were correctly classified by the predictors which was similar to the bacterial infection only group, 27/29 (93.1%). Using a threshold of 0.36 for the clinical predictors and 0.42 for the Suarez genes to calculate naive sensitivity 12 of 14 (85.7%) were correctly categorized by the clinical variables, whereas, 8 of 14 (57.1%) were correctly classified by the model described herein using the 10 genes selected by Suarez et al. There was one subject misclassified as non-bacterial by all 3 models, while all other misclassified subjects differed by model.

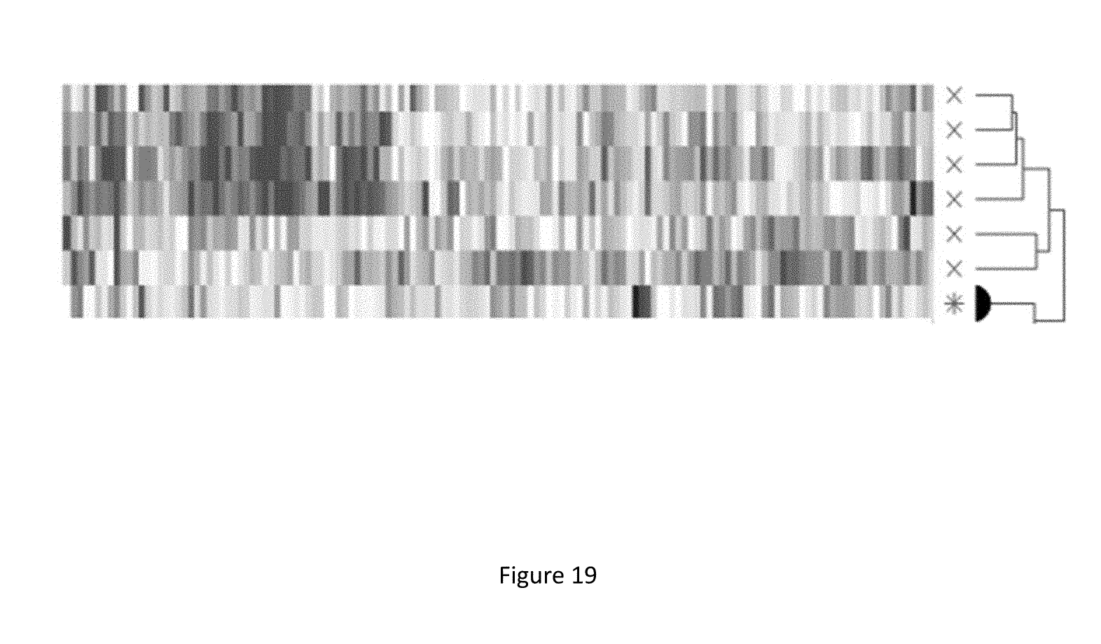

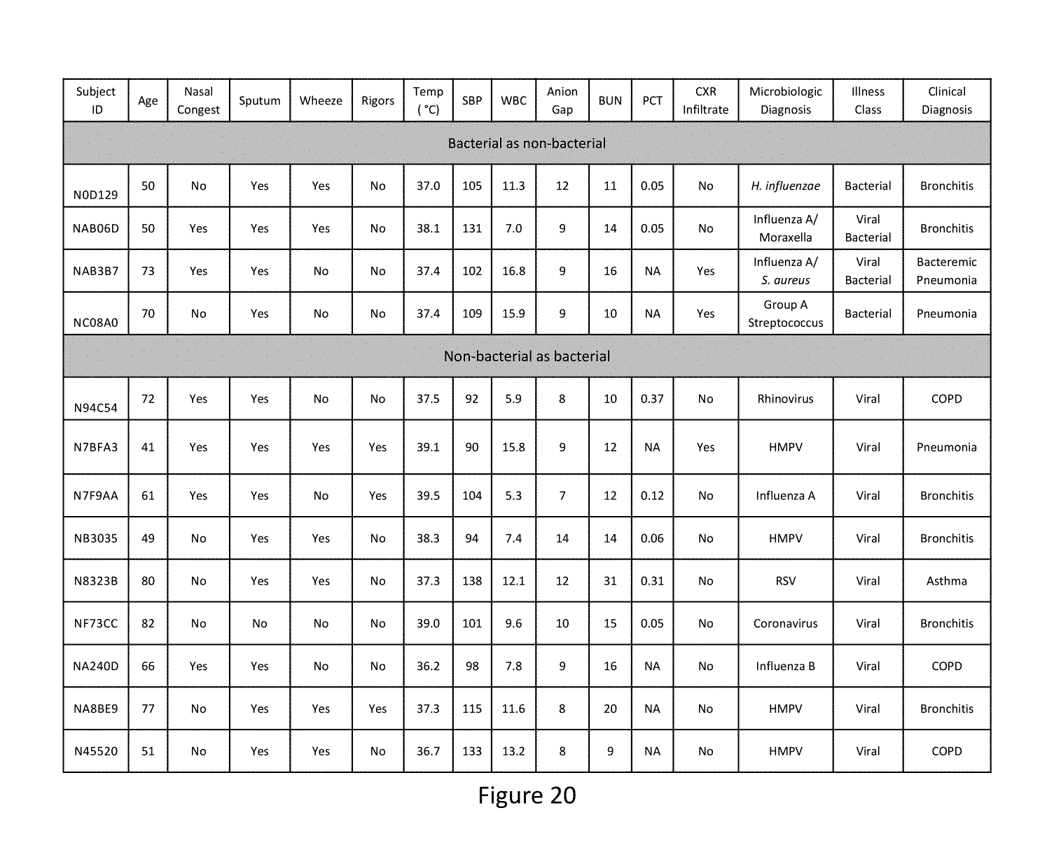

Clinical information on the naively misclassified subjects is provided in FIG. 20. Of note, one subject with influenza A and one of two sets of blood cultures positive for methicillin resistant Staphylococcus aureus (MRSA) was classified as non-bacterial (indicated by the pink semi-circle on the heat map in FIG. 5A and FIG. 5B, and in FIG. 19 (showing only subjects with Staphylococcus aureus infections). Additional clinical information about this subject is provided in FIG. 19.

Biomarker signatures for bacterial infection

Respiratory infections are common reasons for hospitalization in adults and although broad-spectrum antibiotics are frequently prescribed, this practice is now being questioned (Branche et al., 2015, J Infect Dis 212:1692-700; Caliendo et al., 2013, J Infect Dis 57:S139-70). While progress has been made in viral detection, the inaccessibility of the primary site of bacterial infection (the bronchi and lung) makes accurate bacterial diagnostics difficult to develop. Because blood samples can be obtained in most patients, identifying circulating biomarkers reflecting pathologic processes in the lower airways is highly desirable (Zaas et al., 2014, Trends Mol Med 20:579-88 ; Zumla et al., 2014, Lancet Infect Dis 14:1123-35 ; Blasi et al., 2010, Pulm Pharmacol Ther 23:501-7). A variety of protein biomarkers including C-reactive protein and pro-calcitonin (PCT) have been used singly and in combination to discriminate bacterial from viral infection (Blasi et al., 2010, Pulm Pharmacol Ther 23:501-7; Tsalik et al., 2012, J Emerg Med 43:97-106; Oved et al., 2014, PLoS One 10:e0120012). While PCT has been used with some success to guide antibiotic treatment for LRTI, threshold levels have never been validated with microbiology (Shcheutz et al., 2010, Cochrane Database Syst Rev. CD007498; Fowler et al., 2011, Clin Infect Dis 52:S351-6).

As an alternative to serum protein biomarkers, gene expression analyses using peripheral blood have been used in cancer, cardiovascular, autoimmune and infectious diseases to study disease pathogenesis, severity and recently as a diagnostic tool (Chaussabel et al., 2010, BMC Biol 8:84; Tsalik et al., 2016, Sci Transl Med 8:322ra311; Chaussabel et al., 2008, Immunity 29:150-64; Heinonen et al., 2016, Am J Respir Crit Care Med 193:772-82; Rose et al., 2015, PLoS One 10:e0132259). Microarrays have been used to determine unique host response expression “signatures” for tuberculosis, malaria, bacterial and viral infections (Ramilo et al., 2007, Blood 109:2066-77; Hu et al., 2013, PNAS 110:12792-7; Suarez et al., 2015, J Infect Dis 212:213-22; Tsalik et al., 2016, Sci Transl Med 8:322ra311; Anderson et al., 2014, NEJM 370:1712-23; Hu, 2016, Asian Pac J Trop Med 9:313-23). These signatures have been used to differentiate viral from bacterial disease infection, symptomatic from asymptomatic viral infections and to identify specific bacterial and viral pathogens (Heinonen et al., 2016, Am J Respir Crit Care Med 193:772-82; Hu, 2016, Asian Pac J Trop Med 9:313-23).

Several studies have evaluated gene expression by microarray for diagnostic purposes in adults and children with ARI and febrile illness. Interestingly, despite similar accuracy of predictive gene sets (AUC ranging from 78-94%), there has been little overlap in predictive genes identified (Ramilo et al., 2007, Blood 109:2066-77; Hu et al., 2013, PNAS 110:12792-7; Suarez et al., 2015, J Infect Dis 212:213-22; Tsalik et al., 2016, Sci Transl Med 8:322ra311; Zas et al., 2013, Sci Transl Med 5:203ra126; Zaas et al., 2009, Cell Host Microbe 6:207-17; Parnell et al., 2012, Crit Care 16:R157; Herberg et al., 2016, JAMA 316:835-45; Mahajan et al., 2016, JAMA 316:846-57). Diverse populations and control groups studied plus alternate analytic tools used likely explain the different predictive genes identified. Developing a model with the goal of “ruling out bacterial infection” such as the model described herein might be expected to yield different results than those with a goal of identifying influenza regardless of bacterial status (Zaas et al., 2009, Cell Host Microbe 6:207-17; Parnell et al., 2012, Crit Care 16:R157; Statnikov et al., 2010, Cell Host Microbe 7:100-1). Recently, Tsalik used micro array to assess gene expression in whole blood to discriminate bacterial from viral infection or non-infectious cardiopulmonary illness in 273 subjects with community onset ARI (Tsalik et al., 2016, Sci Transl Med 8:322ra311). These investigators used sparse logistic regression to define 130 predictor genes in a model with an accuracy of 87% to discriminate clinically adjudicated bacterial, viral, and non-infectious illness.

One of the goals of this study was to prospectively validate the 10 predictor genes identified by Suarez in an independent cohort of hospitalized adults (Suarez et al., 2015, J Infect Dis 212:213-22). As described herein, differential expression of all 10 genes was confirmed. In addition, additional discovery efforts was included since RNAseq has not previously been used for diagnostic purposes in LRTI. A number of new differentially expressed genes were discovered. Interestingly, in contrast to prior studies which detected genes increased in non-bacterial LRTI, most of the novel genes show increased expression in bacterial LRTI. Of note, subjects were enrolled at the same community hospital and infection status determined using the same criteria as in the Suarez study. In addition to technical differences in microarray and RNAseq methods, predicting bacterial vs. non-bacterial rather than distinguishing viral, bacterial and mixed viral/bacterial infections may have reduced the prominence of interferon related genes identified in the current study. Regardless, these methods for interpretation of differentially expressed genes (FIG. 8, FIG. 14 and FIG. 18) clearly predict the involvement of viral responses and activation of interferon signaling.

The use of clinical variables, even in combination with laboratory variables, has not successfully discriminated bacterial from viral infection with sufficient precision to be useful (Muller et al., 2007, BMC Infect Dis 7:10). Although the predictive accuracy of the clinical variable model was almost as high as this model using gene expression data, this is likely explained by over representation of “extreme phenotypes”. To avoid misclassification, patients with bacteremic pneumonia and virally induced asthma exacerbations represented a substantial proportion of the subjects in this study.

In order for gene expression analysis to move from bench to clinic, a optimal predictive genes should be identified for which rapid PCR can be performed, and thresholds should be chosen to categorize patients as bacterial or non-bacterial. The molecular classifier reported here displays good sensitivity and specificity for predicting bacterial involvement in LRTI. The challenging task of classifying mixed viral bacterial infections as bacterial is described herein. Since many clinicians currently use PCR testing to diagnose viral infections but prescribe antibiotics despite positive results because of fear of bacterial co-infections the ability to correctly categorize this group is important. (Shiley et al., 2010, Infect Control Hosp Epidemiol. 31, 1177-1183; Falsey et al., 2013, J Infect Dis 207:432-41) The current default position is to prescribe antibiotics to hospitalized patients with respiratory infection. One patient in this study with influenza A pneumonia and a single blood culture positive for MRSA was classified as non-bacterial using this pathway-based gene model. In clinical practice blood isolates of S. aureus are almost never dismissed as a contaminant although the organism is a skin commensal. Interestingly, the gene expression pattern of this patient is markedly different than other patients with S. aureus bacteremia raising the possibility that the blood culture was a contaminant (FIG. 5B and FIG. 19).

Thus, all available data was used to build the best final model, rather than setting aside a substantial portion as a validation data set. However, cross-validation was used to avoid over-fitting the training data. Moreover, in a rigorous and computationally intensive move, an outer loop of nested cross-validation was used as an unbiased method to evaluate overall performance of the internally cross-validated model fitting procedure. Compared with data splitting, nested cross-validation results in better models and more stable estimates of performance, since every subject contributes to the performance estimates. Strengths of the current study is the use of high throughput sequencing to classify bacterial and non-bacterial subjects, representing a novel unbiased diagnostic approach as well as inclusion of mixed viral bacterial infections in the predictive model.

In conclusion, this report adds to mounting evidence that gene expression analysis of peripheral blood can be a useful test to discriminate bacterial and nonbacterial respiratory illness in hospitalized adults.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for treatment of a respiratory infection in a subject comprising a step of administering to the subject, who has been identified as having a differentially expressed level of one or more biomarkers selected from one or more biomarkers set forth in Table 1, an effective amount of an antibiotic.

2. The method of claim 1, wherein the one or more biomarkers is an RNA biomarker.

3. The method of claim 1, wherein the one or more biomarkers is selected from the group consisting of ICAM1, ITGAL, ITGB2, PECAM1, FADS2, PLA2GA4, CTSG, IGFBP2, IGFBP6, MMP2, ACOX3, and any combination thereof

4. The method of claim 1, wherein the respiratory infection is a lower respiratory tract infection.

5. The method of claim 1, wherein the antibiotic is selected from the group consisting of Amoxicillin, Ampicillin, Cloxacillin, Dicloxacillin, Nafcillin, Oxacillin, Penicillin G, Penicillin V, Piperacillin, Cefadroxil (cefadroxyl), Cefalexin (cephalexin), Cefalotin (cephalothin), Cefapirin (cephapirin), Cefazolin (cephazolin), Cefradine (cephradine), Cefaclor, Cefotetan, Cefoxitin, Cefprozil (cefproxil), Cefuroxime, Cefdinir, Cefixime, Cefotaxime, Cefpodoxime, Ceftizoxime, Ceftriaxone, Ceftazidime, Cefepime, Ceftobiprole, Ceftaroline, Aztreonam, Imipenem, Imipenem, cilastatin, Doripenem, Meropenem, Ertapenem, Azithromycin, Erythromycin, Clarithromycin, Dirithromycin, Roxithromycin, Clindamycin, Lincomycin, Amikacin, Gentamicin, Tobramycin, Ciprofloxacin, Levofloxacin, Moxifloxacin, Trimethoprim-Sulfamethoxazole, Doxycycline, Tetracycline, Vancomycin, Teicoplanin, Telavancin, and Linezolid.

6. A method of diagnosing a bacterial infection in a subject, the method comprising:
a. detecting the level of one or more biomarkers in a biological sample obtained from the subject, wherein the one or more biomarkers is selected from one or more biomarkers set forth in Table 1;
b. comparing the level of the one or more biomarkers in the biological sample to a control level of the one or more biomarkers; and
c. diagnosing the subject with a bacterial infection when the one or more biomarkers is differentially expressed in the biological sample as compared to the control level.

7. The method of claim 6, wherein the one or more biomarkers is selected from the group consisting of ICAM1, ITGAL, ITGB2, PECAM1, FADS2, PLA2GA4, CTSG, IGFBP2, IGFBP6, MMP2, ACOX3, and any combination thereof

8. The method of claim 6, wherein the biological sample is selected from the group consisting of blood, plasma, saliva, and urine.

9. The method of claim 6, wherein the subject is diagnosed when the one or more biomarkers is increased as compared to the control level.

10. A kit for diagnosing a bacterial infection, the kit comprising a reagent for measuring the level of one or more biomarkers in a biological sample of a subject, wherein the one or more biomarkers is selected from one or more biomarkers set forth in Table 1.

11. The kit of claim 10, wherein the one or more biomarkers is selected from the group consisting of ICAM1, ITGAL, ITGB2, PECAM1, FADS2, PLA2GA4, CTSG, IGFBP2, IGFBP6, MMP2, ACOX3, and any combination thereof.

12. The kit of claim 10, wherein the biological sample is selected from the group consisting of blood, urine, saliva and plasma.
